(19) United States
(12) Patent Application Publication
(10) Pub. No.: US 2006/0270935 A1
(43) Pub. Date: Nov. 30, 2006

(54) Ultrasonic periodontal device and method of using

(75) Inventors: Gregory D. Ariff, Herndon, VA (US); Jennifer L. Case, Alexandria, VA (US); Robert Jason Gwaltney, Springfield, VA (US); Chistian Haller, Alexandria, VA (US); Patrick Hardin, St. Augustine, FL (US); Jeffrey Cameron Loper, Alexandria, VA (US); Charles Luddy, Alexandria, VA (US); Lynessa Erler, Alexandria, VA (US); Jeffrey Smithanik, Calgary (CA); Andrew Steinberg, Arlington, VA (US); Craig Swanner, Alexandria, VA (US)

Correspondence Address: PATTON BOGGS LLP, 8484 WESTPARK DRIVE, SUITE 900, MCLEAN, VA, 22102, US

(73) Assignee: Perioimaging, Inc.

(21) Appl. No.: 11/393,688

(22) Filed: Mar. 31, 2006

Related U.S. Application Data
(60) Provisional application No. 60/666,631, filed on Mar. 31, 2005.
(60) Provisional application No. 60/669,003, filed on Apr. 7, 2005.

(51) Int. Cl. A61B 8/00

(52) U.S. Cl. 600/437; 600/438; 600/449

(57) ABSTRACT

System and methods of detecting and measuring periodontal disease comprising filling a periodontal pocket with a fluid capable of propagating sound waves, transmitting a sound wave into the periodontal pocket, sensing the return sound wave from the periodontal pocket, and determining the depth of the pocket by measuring the time it takes the at least one transmitted sound wave to traverse the periodontal pocket and return. A peak discrimination analysis algorithm is also provided.

### FIELD OF THE INVENTION

The present invention is generally directed to the field of periodontal medicine and in particular to the application of ultrasonic technology to periodontal medicine and to general dentistry.

### BACKGROUND OF THE INVENTION

Periodontal gum disease is a serious infection of the mouth that, if left untreated, can lead to tooth loss and has been associated with, and is suspected of contributing to heart attacks, strokes, diabetes, respiratory diseases, premature/underweight babies and even death.

Periodontal disease can affect one tooth or many teeth. It begins when the bacteria in plaque (the sticky, colorless film that constantly forms on everyone's teeth) causes the gums to become infected and inflamed.

In the mildest form of the disease, gingivitis, the gums redden, swell and bleed easily. There is usually little or no discomfort. Gingivitis is often caused by inadequate oral hygiene, especially lack of flossing. Gingivitis is reversible with professional treatment and good oral home care.

Untreated gingivitis can advance to periodontitis. With time, plaque can harden into calculus and spread and grow below the gum line where it can become a breeding ground for bacteria below the gum line. Toxins produced by the bacteria in plaque and calculus continue to irritate and inflame the gums and surrounding tissue. As the infection becomes more severe, the toxins stimulate a chronic inflammatory response in which the body in essence turns on itself and the tissues (ligaments) and bone that support the teeth are broken down and destroyed.

Periodontal soft tissue (gums or gingiva and the periodontal ligament) detach from the teeth, forming periodontal pockets (spaces between the teeth and periodontal tissue) that become infected. As the disease progresses, more and more destructive toxins are produced and as a result, the periodontal pockets deepen and more periodontal tissue and bone are broken down and destroyed. Initially this destructive process may be asymptomatic. Eventually, teeth can become loose and may be lost or have to be removed. More than 300 different types of bacteria can exist in the human mouth, either alone or in combination. This makes treating periodontal disease difficult, time consuming and expensive as the periodontist tries various antibiotics and treatment modalities until an effective treatment plan is developed. Like any other serious infection, if not promptly treated with the proper types and amounts of antibiotics, periodontitis can result in severe systemic infection that can lead to many other serious diseases and even become life-threatening. As the patient's immune system fights this chronic and perhaps serious infection, it can create an opportunity for other serious diseases, such as heart disease, stroke and diabetes, to develop.

The current methodology used by dentists and dental hygienists to detect and measure periodontal pocket depth is a primitive methodology that consists of a sharp metal probe that is inserted between the tooth and gum and which is manually pressed down until it encounters resistance of the ligament. The depth to the ligament is thereby measured and indicates the amount of clinical attachment lost (loss of ligament), which can be an indication of the amount of periodontal disease that may be present. This method is often painful for the patient, and is invasive, bloody, inaccurate and subjective. It is especially inaccurate and subjective because of the difficultly in applying the same amount of force with each measurement, resulting in high intra-examiner and inter-examiner variation in measurement. The difficulty is increased because the examiner does not know the type of tissue present below the gum line and if the probe is touching or piercing this tissue. Additionally, exposure to the patient's blood by dental professionals increases risk of exposure to hepatitis, HIV and other infectious diseases.

Further, the current methodology is limited in its effectiveness as a tool for diagnosing periodontal disease in its earliest stages as it is a retrospective analysis and can only measure significant amounts of tissue already lost. In addition, this method typically calls for two people to perform this test, an examiner who actually makes the measurements and a scribe who usually writes down the measurements by hand. The examiner is generally a dental health professional, such as a dentist, dental hygienist or periodontist. The scribe may also be a dental health professional but may also be a lesser skilled individual such as an office assistant. Another problem facing dentists is the difficulty in determining long term trends of the patients' condition because all of the information is contained in numerous paper (i.e., analog) records that usually span many years. As a result, usually only the last one or two records are reviewed for comparison with the current test results and these may not be sufficient to accurately reflect a very gradual deterioration of the patient's periodontal condition.

An additional problem with the existing manual probe methodology is that it is typically can be disruptive to the healing process. The trial and error approach can tear newly healed tissue and can cause recovery to be extended for weeks or months. Further, it can allow bacteria into the wound and the patient's blood stream, which can lead to infection (i.e., bacteremia). Indeed more than 300 different types of bacteria can exist in the human mouth, either alone or in combination. This makes treating periodontal disease difficult, time consuming and expensive as the periodontist tries various antibiotics and treatment modalities until an effective treatment plan is developed.

FIG. 1 is a schematic diagram comparing a healthy tooth 100 on the left and a tooth 106 with periodontal disease on the right. The healthy tooth 100 has a full, healthy bone level 104, healthy periodontal ligament 103, and a healthy gum/gingiva 102. The diseased tooth 106 exhibits gum/gingiva loss 116, loss of periodontal ligament attachment (clinical attachment loss) 115 and resorption of avelor bone level 114, resulting in the formation of a periodontal pocket 112. The diseased tooth 106 also exhibits a build up of plaque 108 and tartar/calculus 110. If the periodontal condition is not diagnosed and corrected, the diseased tooth 106 may be lost or have to be removed.

FIG. 2 is a more detailed schematic diagram of the teeth 100, 106 illustrated in FIG. 1. The teeth 100, 106 have an enamel portion 118 and a root portion 120. The root portion 120 is connected to the gum 122 by the periodontal ligament 126. The top of the gum 122 is known as the gum line 124. As illustrated in FIG. 2, the gum line 124 has receded. In some cases, however, the gum 122 may be irritated, resulting in the gum line 124 rising due to edema.

At the top of the periodontal ligament 126 is the upper boundary 130 of the periodontal ligament 126. Between the upper boundary 130 of the periodontal ligament 126 and the enamel portion 118 is the junction epithelium 128. In a healthy tooth 100, the upper boundary 130 of the periodontal ligament 126, the bottom of the junction epithelium 128 and the enamel portion 120 meet at the cemento-enamel junction 132. In a diseased tooth 106, tartar or calculus 110 and polymorphonuclear leukocytes 138 spread into the junction epithelium 128 and the periodontal ligament 126 opening a periodontal pocket 112. If the periodontal pocket 112 lies between the gum line 124 and the cemento-enamel junction 132, the patient has a condition known as gingivitis. If the periodontal pocket 112 extends below the cemento-enamel junction 132, the patient has a condition known as periodontitis. Additionally, the growth of the periodontal pocket 112 may be irregular and result in intermediate features 136.

Frequently, prior measurements of pocket depth were made relative to the gum line 124. As discussed above, however, the gum line 124 may vary due to recession or edema. Therefore, use of the gum line (or free margin of gingiva) 124 in measuring pocket depth may lead to inaccurate and widely varying measures of pocket depth. In contrast to the gum line 124, the location of the cemento-enamel junction 132 remains constant. Therefore, use of the cemento-enamel junction 132 in measuring pocket depth provides a better and more consistent method of measurement over time and is preferable. Manual probing is also used to determine if, and on which teeth and exactly where, calculus is present below the gum line. This method can be inaccurate.

It would therefore be desirable to have a painless, noninvasive, accurate and reproducible method of measuring periodontal attachment loss capable of using both the gum line and the cemento-enamel junction 132 as a reference. It would also be desirable to have an accurate method of determining if, and on which teeth calculus is present below the gum line.

### SUMMARY OF THE INVENTION

This invention relates to a system for detecting and measuring attachment loss, an indicator of periodontal disease. The invention incorporates the use of ultrasonic technology to measure the differential depth between both the gum line and the cemento-enamel junction of a tooth and the bottom of a periodontal pocket. In contrast to conventional methods that require inserting a sharp metal probe between the teeth and the gum, the present invention provides a system and method that is painless and noninvasive, painless, bloodless, accurate, fast, objective and digital.

The present invention provides a system for detecting and measuring periodontal tissue destruction related to periodontal disease comprising a hand piece having an ultrasonic transducer, and an acoustic lens; a controller unit having discrimination analysis software, the discrimination analysis software comprising a wavelet algorithm; and a fluid supply.

The present invention provides a method of detecting and measuring periodontal tissue destruction related to periodontal disease comprising: filing a periodontal pocket with a fluid capable of propagating sound waves; transmitting at least one sound wave into the periodontal pocket; sensing at least one return sound wave from the periodontal pocket; and determining the depth of the pocket by measuring the time it takes the at least one transmitted sound wave to traverse the periodontal pocket and return.

The present invention provides a dental system for detecting and measuring comprising a dental tool having an ultrasonic transducer; and a controller unit having discrimination analysis software, the discrimination analysis software comprising a wavelet algorithm, the dental tool adapted for signal communication with the controller unit, wherein the ultrasonic transducer is adapted to produce an ultrasonic signal capable of being transmitted thorough a fluid signal coupler to produce dental signals for processing by the discrimination analysis software.

The present invention provides a dental method of detecting and measuring comprising providing a dental tool capable of propagating sound waves through a signal coupler; transmitting at least one sound wave through the signal coupler onto a dental feature; sensing at least one return sound wave from the dental feature; and determining a characteristic of the dental feature by measuring the time it takes the at least one transmitted sound wave to return.

A hand piece for detecting and measuring periodontal disease comprising a continuously curved handle; an ultrasonic transducer; and a fluid supply.

The present invention A method of detecting and measuring periodontal tissue destruction related to periodontal disease comprising providing a hand piece comprising, (i) a permanent handle having a cavity and an alignment slot in a first end, an ultrasonic transducer located in the cavity in the first end of the permanent handle, a fluid supply, and a disposable cover, the disposable cover having a protrusion in the interior of the disposable cover, the protrusion adapted to fit into the alignment slot, or (ii) a hand piece for detecting and measuring periodontal disease comprising a continuously curved handle, an ultrasonic transducer, and a fluid supply; filling a periodontal pocket with a fluid capable of propagating sound waves; transmitting at least one sound wave into the periodontal pocket; sensing at least one return sound wave from the periodontal pocket; and determining the depth of the pocket by measuring the time it takes the at least one transmitted sound wave to traverse the periodontal pocket and return.

The present invention also provides a method of detecting and measuring periodontal disease comprising filling a periodontal pocket with a fluid capable of propagating sound waves; transmitting at least one sound wave into the periodontal pocket; sensing at least one return sound wave from the periodontal pocket; and determining the depth of the pocket by measuring the time it takes the at least one transmitted sound wave to traverse the periodontal pocket and return.

The present invention also provides a method of performing periodontal examinations comprising providing dentists or dental hygienists with at least one ultrasonic periodontal system; and charging the dentist or dental hygienist per examination of a patient.

The present invention also provides a discrimination analysis algorithm to analyze ultrasonic echoes comprising processing waveforms; detecting peaks; and discriminating peaks, wherein the discrimination analysis algorithm uses a continuous wavelet transformation.

### BRIEF DESCRIPTION OF THE DRAWINGS

FIG. 1 is a schematic diagram showing a healthy tooth on the left and one with periodontal disease on the right.

FIG. 2 is a more detailed schematic diagram of the teeth illustrated in FIG. 1.

FIG. 3 is a perspective view of a periodontal system according to a first embodiment of the invention.

FIG. 4 is alternative a periodontal system in accordance with the principles of the invention.

FIG. 5 is a perspective view of a system illustrating one aspect of the invention.

FIG. 6 is a perspective view of a system illustrating another aspect of the invention.

FIG. 7 is a perspective view of a system illustrating another aspect of the invention.

FIG. 8 is a schematic illustration of a disassembled hand piece according to one embodiment of the invention.

FIG. 9 is a schematic illustration of an assembled hand piece illustrated in FIG. 8.

FIG. 10 is a schematic illustration of an embodiment of the invention having an acoustic lens.

FIG. 11 is a functional layout of an embodiment of the invention having foot pedal controls.

FIG. 12 is a schematic illustration of an embodiment of the invention illustrating the use of an enterprise portal.

FIG. 13 is a schematic illustration of a disassembled hand piece according to one embodiment of the invention.

FIG. 14 is a schematic illustration of an assembled hand piece illustrated in FIG. 13.

FIG. 15 is a schematic illustration of a software layout of the invention.

FIG. 16 is flow diagram illustrating operational modes of an embodiment of the invention.

FIG. 17 is flow diagram illustrating screen flows of an embodiment of the invention.

FIG. 18 is a screen shot of an embodiment of the invention, initial login screen.

FIG. 19 is a screen shot of an embodiment of the invention, main screen.

FIG. 20 is a screen shot of an embodiment of the invention, account screen.

FIG. 21 is a screen shot of an embodiment of the invention, options screen.

FIG. 22 is a screen shot of an embodiment of the invention, patient records screen.

FIG. 23 is a screen shot of an embodiment of the invention, patient chart screen.

FIG. 24 is a screen shot of an embodiment of the invention, enter tooth condition data screen.

FIG. 25 is a screen shot of an embodiment of the invention, measure pocket depth screen.

FIG. 26 is a screen shot of an embodiment of the invention, calibration screen.

FIG. 27 is a screen shot of an embodiment of the invention, view tooth history screen.

FIG. 28 is a screen shot of an embodiment of the invention, view full patient chart screen.

FIG. 29 is a screen shot of an embodiment of the invention, patient history selection screen.

FIG. 30 is a screen shot of an embodiment of the invention, help screen.

FIG. 31 is a flow chart illustrating data acquisition and analysis.

FIG. 32 is a flow chart illustrating data entry operator options.

FIG. 33 is a schematic illustration of the external interface arrangement.

FIG. 34 is a schematic illustration of a head and tip portion of a hand piece of an embodiment of the invention.

FIG. 35 is a plot illustrating the effect of flow rate on ultrasound measurements.

FIG. 36 is a plot illustrating a Mexican Hat wavelet.

FIG. 37 is a plot illustrating a Morlet wavelet.

FIG. 38 is a plot illustrating wavelet scale to signal frequency.

FIG. 39 is a plot illustrating peak discrimination.

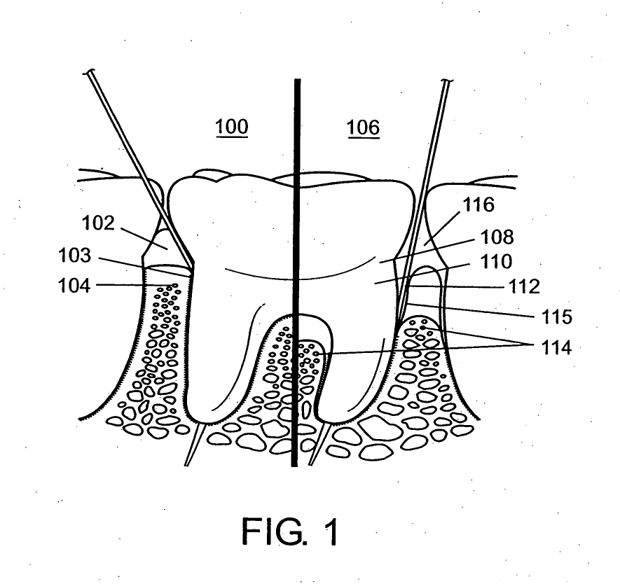

### DETAILED DESCRIPTION OF THE INVENTION

In contrast to the conventional method of detecting and measuring periodontal attachment loss, the systems and methods according to the present invention incorporate ultrasound technology. The systems and methods provides dentists and their patients with a painless, non-invasive, bloodless, extremely accurate, objective, automated, rapid, digital and inexpensive method to effectively diagnose, detect, and evaluate attachment loss related to periodontal disease, create a digital dental record and monitor treatment via a sequence of measurements. The system takes analog measurements, converts the analog ultrasonic measurements to digital data and calculates the periodontal pocket depth (preferably, the distance from both the gum line and the cemento-enamel junction to the upper boundary of the periodontal ligament). The methods according to the present invention enable dentists to detect periodontal disease in its earliest stage when it is easy and inexpensive to treat and before the body's immune system is weakened and susceptible to other diseases. It also permits dentists to more easily and effectively clean their patient's teeth by providing qualitative information regarding the presence of calculus (i.e., hardened plaque) present on tooth surfaces below the gum line, before and after cleaning. Additionally, the methods are essentially examiner independent as inter-examiner and intra-examiner variation has been essentially eliminated.

In contrast to prior art methods of diagnosing periodontal disease, the periodontal system of the present invention allows the dentist to digitally overlay the patients' current test and easily and quickly compare it with some, many or all of the prior tests contained in the patients' electronic medical record. Additional benefits of the periodontal system of the present invention include that its test may be performed by only one person (compared to two people) and it typically only takes about four minutes to complete (compared to about ten minutes).

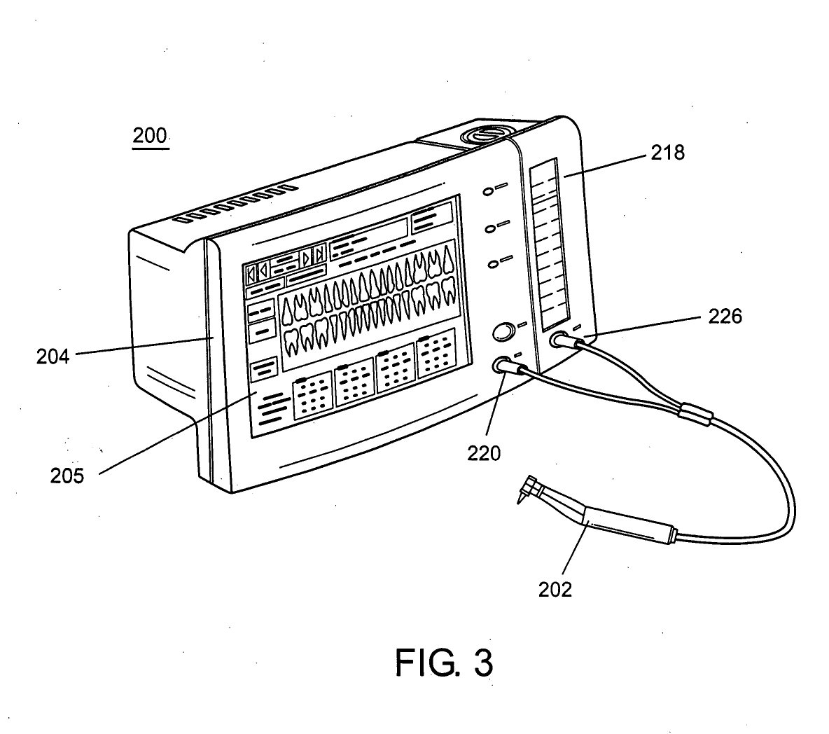

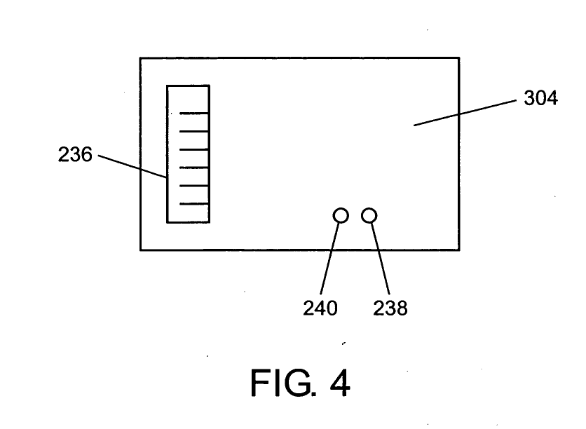

One preferred embodiment of the invention is illustrated in FIG. 3. In this embodiment, the periodontal system 200 includes a hand piece 202 and a display/controller unit 204 (FIG. 4). The display/controller unit 204 includes, circuit boards (not shown) and software to control, acquire, and process the signals, data storage (not shown), a liquid reservoir 236 to hold the liquid used as the signal coupler, at least one liquid flow connector 240, electrical connectors 238, and software and data storage. The display/controller unit 204 is preferably compact, yet includes a large touch-screen 205. Because the display/controller unit 204 contains its own liquid reservoir 236, it can be used in locations without a water supply. Additionally, the liquid reservoir 236 need not be located on the side of the display/controller unit 204 as illustrated in the figures. It may be located, for example, on the bottom or in the back of the display/controller unit 204. Further, for purposes of this disclosure, the word “liquid” encompasses gels.

The software includes an advanced discrimination analysis algorithm. Optionally, it may also include diagnostic medical imaging ability. The periodontal system 200 uses ultrasonic signals (i.e., sonar waves) to detect, quantify and profile the upper boundary 130 of the periodontal ligament 126 (i.e., the depth of each tooth's periodontal pocket 112) below the gum line 124 and from the cemento-enamel junction 132 while also providing qualitative information regarding the presence of calculus or plaque 110, 108 above or below the gum line 124. The periodontal system 200 converts analog ultrasonic signals to digital signals and digitally stores the pocket depths 112 of each tooth 106 and their variation over time. This greatly assists dentists in the diagnosis, and as an indicator of the extent and severity of periodontal disease and the effectiveness of their treatment plan. Preferably, the entire test is fully computerized and all patient information may be digitally recorded by the person performing the test. Preferably, the dentist inputs essential data about each tooth once (e.g., the location of a missing tooth or a bridge), and it will appear on all subsequent screens.

Algorithm

The discrimination analysis algorithm of the periodontal system 200 converts the ultrasonic waveform it receives from the transducer to a pocket depth reading using a transformation algorithm. This algorithm uses signal processing techniques that are commonly used in telecommunications to detect low level signals and isolate them from background noise.

The algorithm is performed in three steps: waveform processing, peak detection, and peak discrimination.

Wavelet Transformation

In one embodiment a Continuous Wavelet Transform (CWT) is performed on the raw signal using the Mexican Hat Wavelet, Ψₛ(x):
Ψ(x)=2πw^{-1/2}[1−2π(x/w)²]e^{-π(}x/w)ˆ2
and
Ψₛ(x)=s^{-1/2}Ψₛ(x/s)
Where:
w, the width of the wavelet, set to 1.2 in one aspect of the invention and
s, the scale of the wavelet, set to the following values: 3.5, 4.21, 5.26, 6.58, and 7.89
   The width and scale of the wavelet were chosen to target the frequency range of the 10 MHz transducer. The wavelet transform of the function f is then equal to:
   W(s,t)=Σf(x)Ψₛ(x−t)
   Wavelet Selection

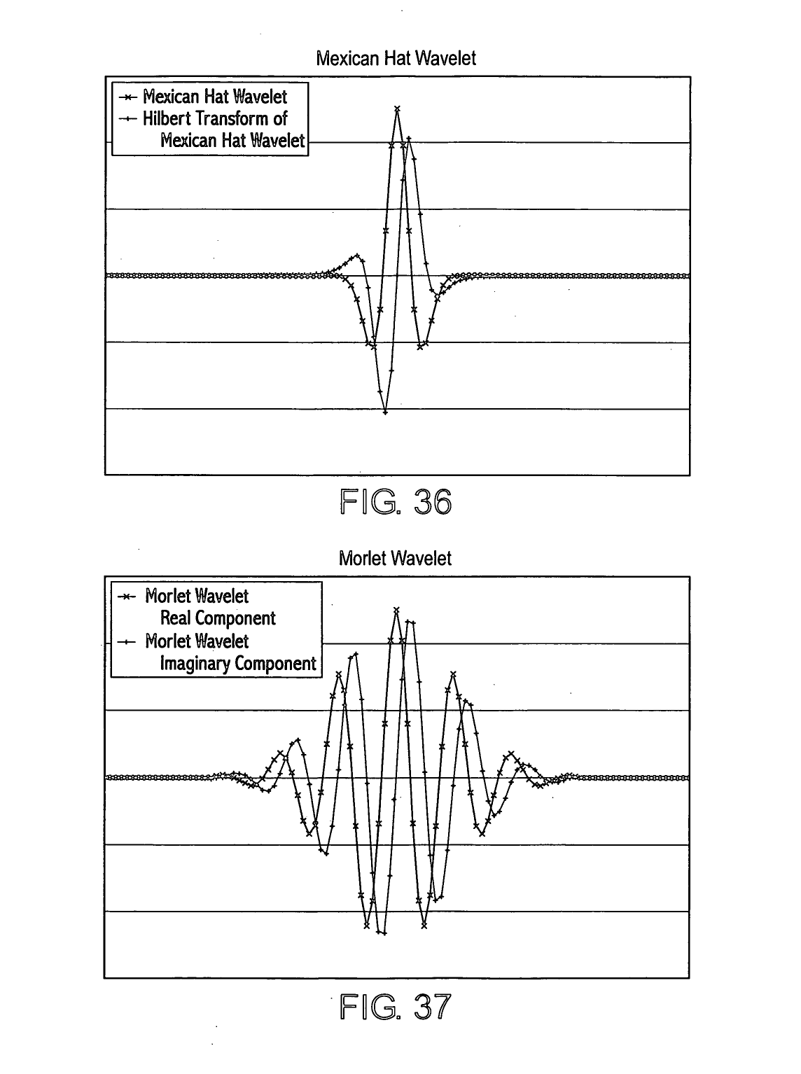

The Mexican Hat wavelet (see FIG. 36) was chosen because, with only 1.5 “oscillations” in the wavelet, it provides better time resolution than wavelets that contain more oscillations, such as the Morlet wavelet (FIG. 37). The trade off is reduced frequency resolution. For the present embodiment of the invention, time resolution is typically more important than frequency, the range of which is fixed by the natural frequency of the transducer crystal.

Unlike many other wavelets used in CWT, the Mexican Hat wavelet does not have an imaginary component. Therefore, to determine the out of phase frequencies present in the raw signal, the wavelet transform is also performed with the Hilbert transform of the Mexican Hat wavelet.

Performing a Hilbert transform on a time based signal generates a π/2 phase shift in the signal. Given a signal g(t), Hilbert transform of this signal is defined as:

$$\hat{g} ( t ) = \frac{1}{π} {\int }_{- ∞}^{+ ∞} \frac{g ( τ )}{t - τ} ⅆ τ$$

Another way to write this definition is to recognize that Hilbert transform is also the convolution of function 1/πt with the signal g(t). The convolution of two functions is the inverse Fourier transform of the product of the Fourier transforms of the two functions:

So we can write the above equation as:

$$\hat{g} ( t ) = \frac{1}{π t} * g ( t )$$

The Fourier transform of 1/πt is:

$$F ( \frac{1}{π t} ) = - jsgn ( f )$$

$$where \text{:}$$

$$sgn ( f ) = \{ \begin{matrix}1 & f > 0 \\ 0 & f = 0 \\ - 1 & f < 0\end{matrix}$$

Therefore, to calculate the Hilbert transform of the Mexican Hat wavelets, first the Fourier transform of the wavelet is calculated. Second, the DC component and the Nyquist frequency component are set to zero. Then the positive harmonics are multiplied by −j and the negative harmonics are multiplied by +j. Finally the inverse Fourier transform is performed on the result to obtain the Hilbert transform of the Mexican Hat wavelets (see FIG. 36).

To increase the processing speed at run-time the wavelet coefficients and transformed wavelet coefficients have been calculated and are preferably coded into the algorithm as constants.

Scale Selection

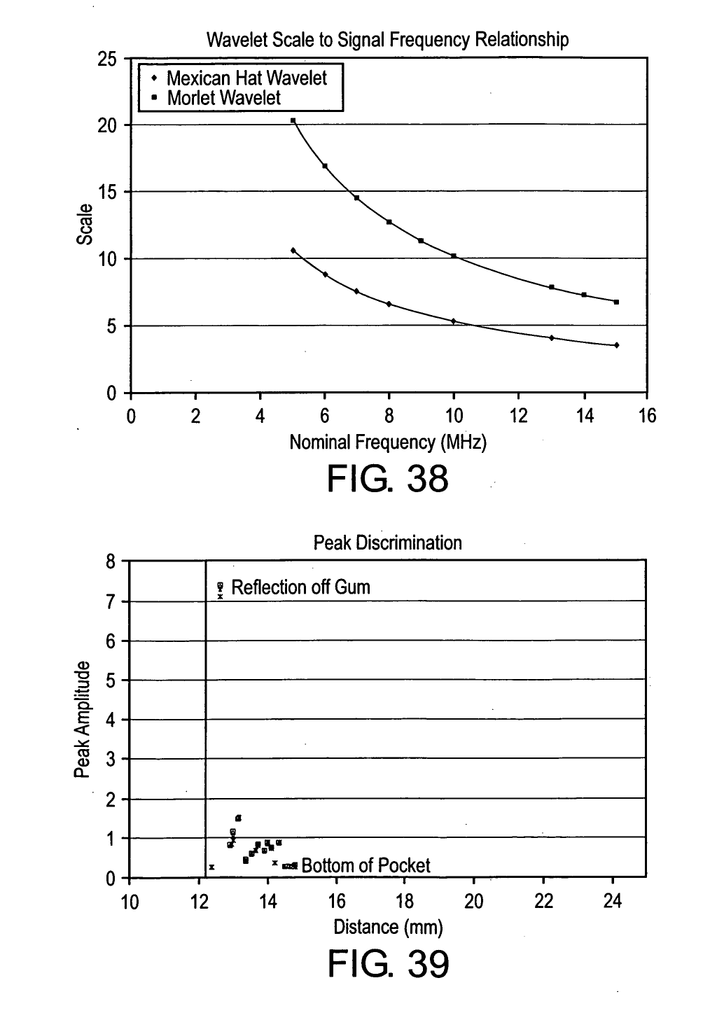

To determine the relationship between the wavelet scale and the signal frequency, sine waves with known frequencies were analyzed to determine the scales that produced the highest wavelet amplitudes. The optimum scale is inversely proportional to the frequency (FIG. 38). A range of scales within the transducer's natural frequency band was selected and the corresponding scales determined (see Table 1).

TABLE 1

| Frequency (MHz) | Mexican Hat Scale |
|---|---|
| 6.66 | 3.5 |
| 8 | 4.21 |
| 10 | 5.26 |
| 12.5 | 6.58 |
| 15 | 7.89 |

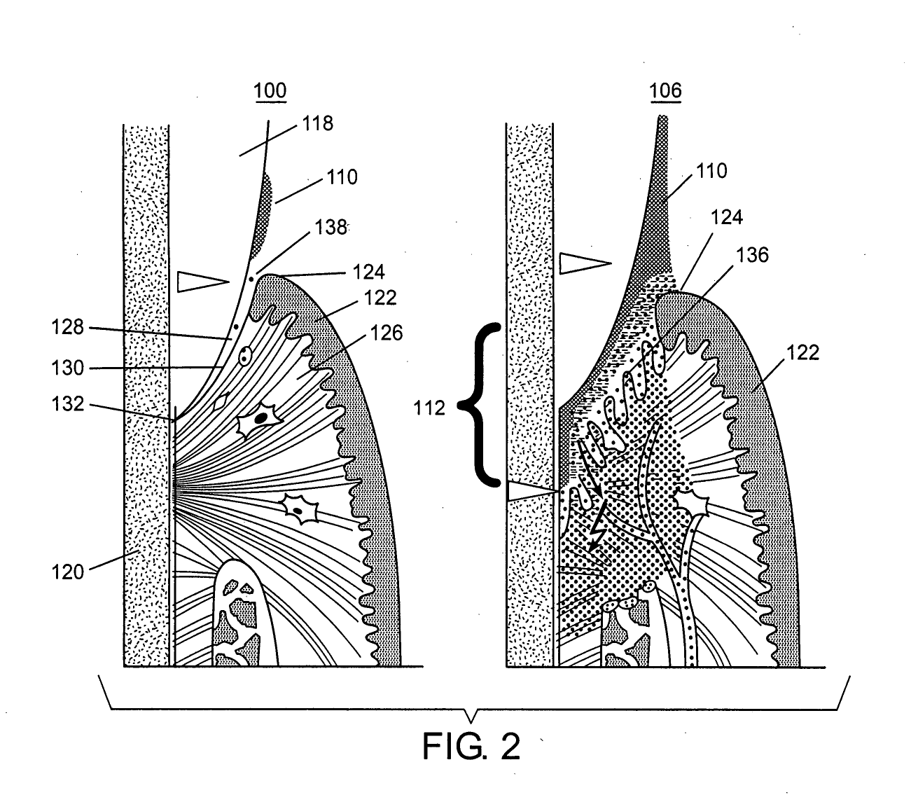

The periodontal system 200 (FIG. 3) may include a digital imaging and diagnostic tool for effectively detecting, mapping, characterizing and evaluating the presence, and monitoring the treatment of periodontal disease. Preferably, it also provides important and useful information regarding calculus (i.e., hardened plaque) 110 (FIG. 2) which harbor bacteria and interferes with dental hygiene present on tooth surfaces below the gum line 124.

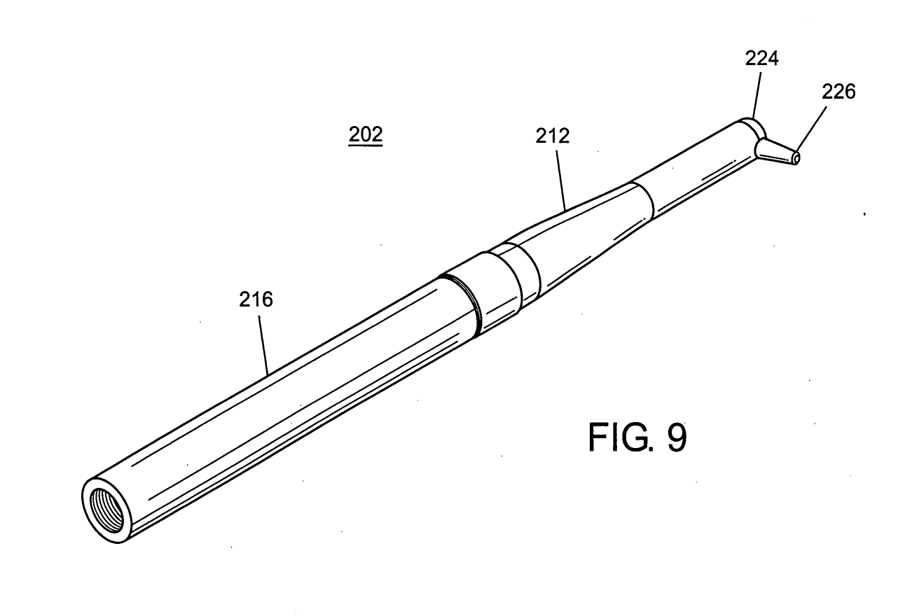

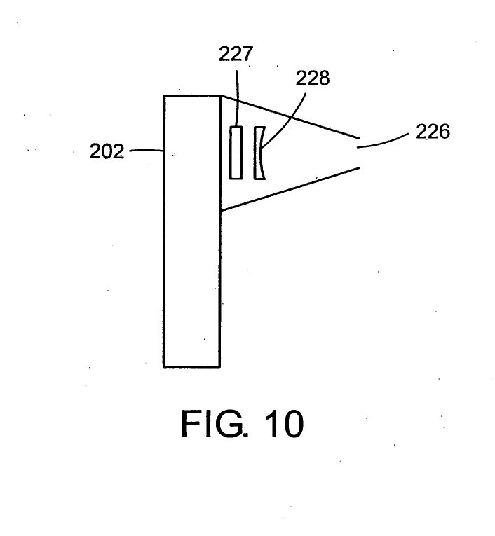

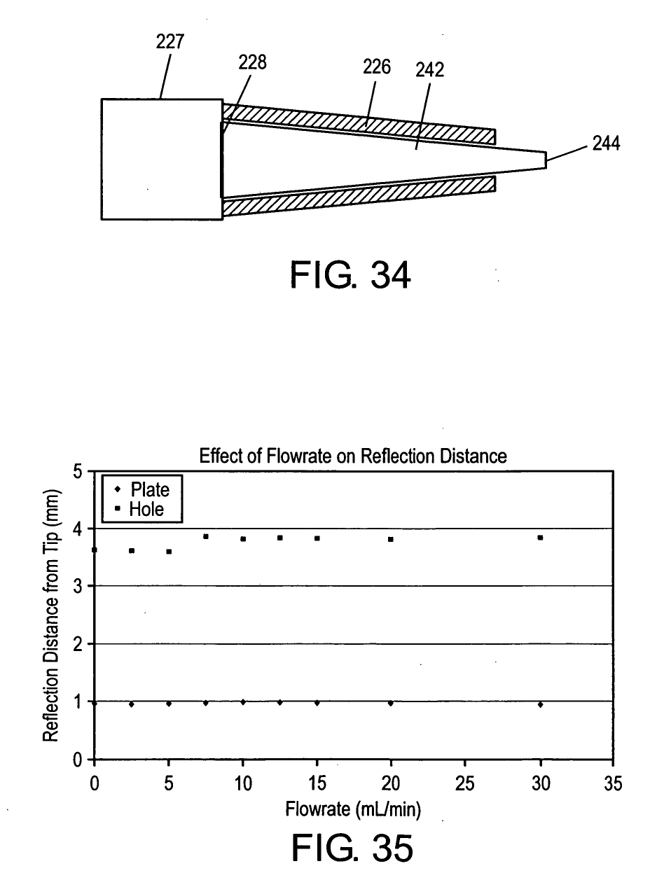

In use, the hand piece 202 (FIG. 9) directs a steady drip or a gentle stream of water onto the gums between the periodontal tissue and the tooth 100, 106. In one embodiment, the dentist then uses one of three buttons on a foot pedal to activate each burst of signals. This permits the dentist to perform the three standard probes on the facial side and the lingual (tongue) side of each tooth. One button advances to the next location, one button activates the signal and if necessary, one button permits the dentist to go back and test the last spot. The dentist is informed with audible tones if a signal was properly obtained or with a different sound if the signal was not properly received (the software recognizes an aberrant reading). The transducer 227 (see FIGS. 10, 34) in the tip of the hand piece 202 transmits ultrasonic signals (i.e., sonar waves) below the gum line 124 (along each tooth's 100, 106 surface and into the periodontal pockets 112), using an anti-bacterial or germicidal gel, applied to the gums, or water (or other liquid such as solution containing an anti-bacterial agent or an germicidal agent) dripped onto the gums as the signal coupler. The signal may also use saliva below the gum line as the signal coupler. The transducer 227 captures the corresponding echoes resulting from their collision with normal and/or abnormal anatomical features below the gum line 124. The time each signal takes to make the round trip is measured. From this measurement, the distance the signal traveled to the feature causing the reflection can be determined. With this information, the system's 200 advanced discrimination analysis algorithm can provide healthcare professionals with a painless, non-invasive, extremely accurate, fast, automated, digital and user friendly method to provide important information regarding the true condition of the patient's periodontal anatomy and on each tooth's surface below the gum-line 124.

In other embodiments of the invention, the display/controller unit 204 (FIG. 3) can be directly connected to an existing water supply. Optionally, the circuit boards can convert the signals into a series of user-friendly images. In one aspect of the invention, the display/controller unit 204 includes a keyboard and mouse rather than a touch screen. In another embodiment of the invention, the system 300 (FIG. 4) includes a controller unit 304. Similar to display/controller unit 204, controller unit 304 includes data storage (not shown), a water reservoir 236, water flow connectors 240, electrical connectors 238 and circuit boards (not shown) to control, acquire and process the signals. In contrast to display/controller unit 204, controller unit 304 does not include a display. In this embodiment of the invention, controller unit 304, is connected to the healthcare professional's existing computer monitor. The connection may be accomplished through a hard wire connection such as through a USB port or wirelessly such as BlueTooth.

Embedded software within the computer converts the signal from an analog to a digital format and uses algorithms to interpret and convert the echoes corresponding to the depth of the outer boundary of the periodontal ligament into a dimension (e.g., pocket depth in millimeters) and to detect the presence of calculus 110 on the tooth's surface, above or below the gum line 124 so it can be more easily and effectively removed.

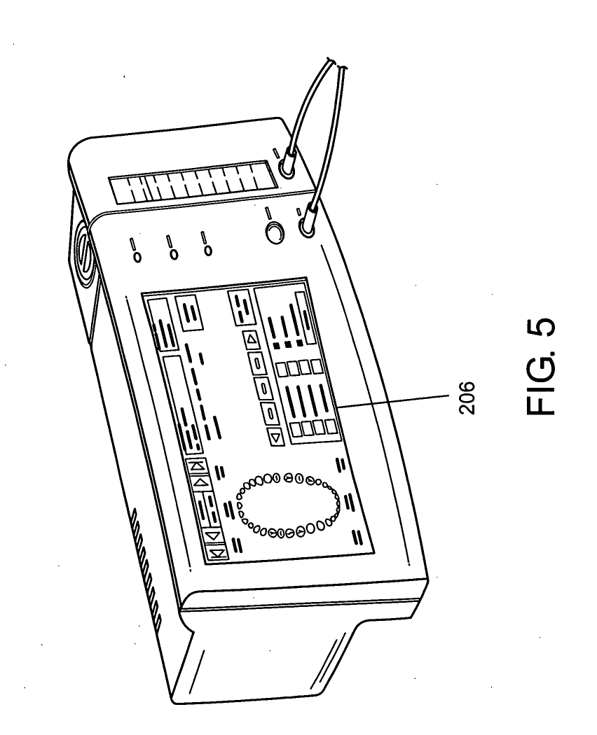

The display/controller unit 204 receives the analog information generated by the hand piece 202, converts the data into a digital format, and processes it using analysis algorithms. Preferably, the periodontal system 200 also includes dental imaging software to and creates user friendly images of the applicable tooth 100, 106 (as shown on the screens in FIGS. 5-7). The images can be displayed on the display/controller unit's 204 large, color touch-screen. If the dentist wants to view the test results on another screen in his operatory, the display/controller unit 204 will transmit the images to the dentist's screen on a wireless or wired basis. The dentist can input all patient information using the display/controller 204 unit's large, user-friendly touch-screen, or a keypad. In another aspect of the invention, the dentist can input all patient information using voice recognition software. Further, the system is easily and quickly moved between operatories using its quick connect/disconnect water and electrical fittings.

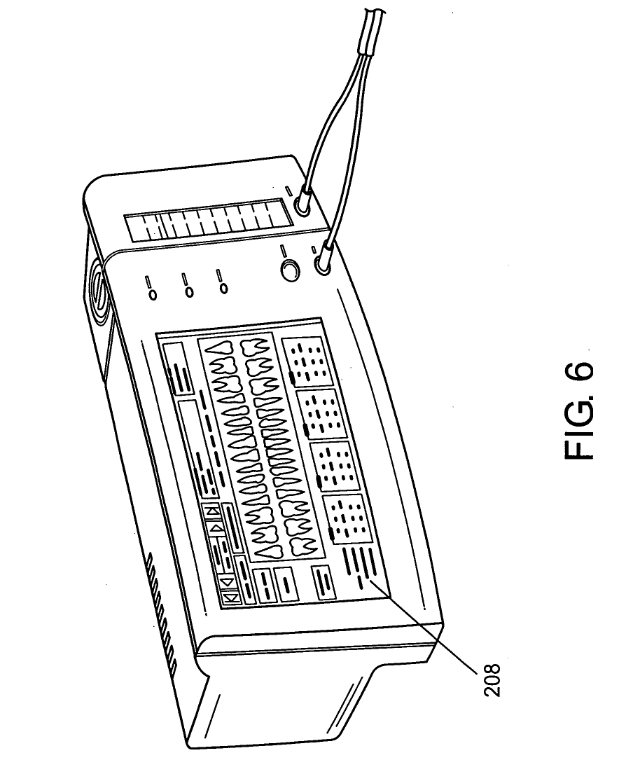

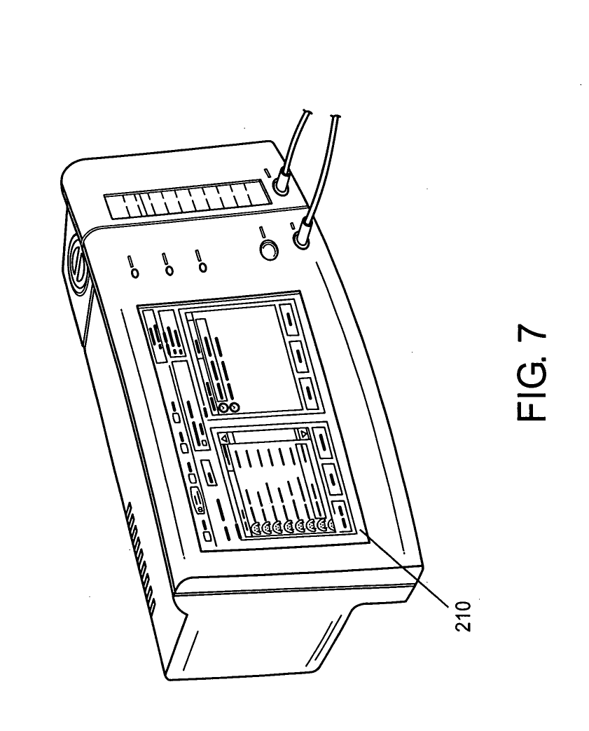

The software preferably included in system 200 (FIG. 3) preferably allows the display/controller unit 204 to display periodontal measurement 206 (FIG. 5), the charting of results 208 (FIG. 6), and patient management 210 (FIG. 7). In one embodiment, the software may generate an image of a tooth with surrounding periodontal tissue and illustrate the data on the image of the tooth. In this way, healthcare professionals and patient's can visually monitor the progress and/or treatment of periodontal disease and/or the removal of calculus below the gum line.

Preferably, the periodontal system 200 is calibrated before examining each patient. Calibration may include testing the software, calibrating the head, testing the transducer and/or testing the acoustic lens. Calibration may be accomplished, for example by measuring the depth of a known cavity built into the unit 204. Should the periodontal system 200 fail such that recalibration in the healthcare provider's office is not possible, the system 200, may optionally be provided with automatic messaging that can transmit a request for a new system 200, or part thereof, from the supplier.

In one embodiment of the invention, all tests will be performed after the dentist or dental hygienist connects to a third party web site via the Internet. This will permit the third party to confirm the periodontal system 200 is properly calibrated and working perfectly prior to each test and that the dentist's account has been properly charged the test fee. Patient information may be securely stored in a HIPAA compliant centralized back-up database maintained by the third party at its website. In this embodiment, dentists will have controlled access to the website and be able to:
Review their patient records;
Review and update their account information;
Review and update their disposable inventory and ordering information; and
Review the status of the system(s) in their office.

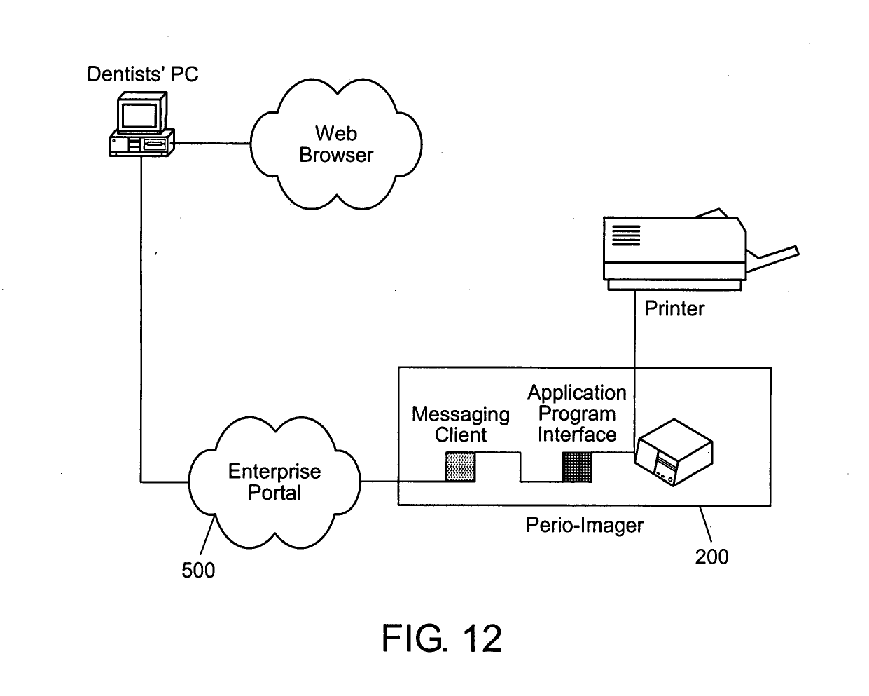

In one embodiment of the invention, illustrated in FIG. 12, the connection to the third party is accomplished with enterprise portal software 500. In this embodiment, dental records can be stored at the third party site for backup purposes. Further, dental records (including their digital images) can be forwarded to periodontal experts for online consultation. The periodontal expert can evaluate the patient's condition and send his evaluation and recommendations either to the third party or directly to the dentist or dental hygienist the patient is seeing. Further, the enterprise software is capable of monitoring the dentist's activities. The system can be used to determine which dentists are successful in treating minor periodontal problems and those who are not. Further, it can be used to identify dentists who are treating patients that should be under the care of a periodontist due to the severity of the patient's condition. Additionally, software for the periodontal system 200 can be easily upgraded via a simple download by request by the user upon notification that an update is available or automatically by the provider if a service agreement is in place. In still another embodiment, the dental records may be encrypted.

The system permits dentists to show the test results on their computer screens (rather than on paper records from multiple years) to their patients and also provides them with a printout of the test. This permits patients to confirm what their dentists have told them and to monitor the effectiveness of their treatment plan. This active patient involvement is expected to result in more patients following their dentist's instructions because they will be able to see that their periodontal disease treatment program is working. It also provides patients (and their payers) with objective proof of the presence of periodontal disease and the necessity of treatment. This is expected to reduce the number of “walkaways” (i.e., patients that do not believe their dentist or the severity of their periodontal condition).

If a patient does not currently have periodontal disease, by comparing the base line digital images with those taken over a period of time, both the dentist and the patient can see that periodontal disease is not present.

According to one embodiment of the invention, periodontal examiners establish a baseline of their patient's periodontal pocket depths 112 (FIG. 2) during an initial exam. After their first examination, each time the patient has a new examination, the data from the prior examinations may be digitally and automatically compared to the current data and illustrated with the periodontal system's 200 dental display software. This permits dentists and their patients to identify even relatively minor changes in periodontal pocket depths 112 not otherwise detectable using the current manual probe method. These minor changes may be illustrated with color trend lines that reflect improving, deteriorating or unchanged pocket conditions. In one aspect of the invention, changes of approximately 0.2-0.5 mm may be detected. In another aspect of the invention, changes of approximately 0.1-0.2 mm may be detected. This permits treatment to start while the periodontal disease is in its earliest stages and easily and inexpensively treated.

The periodontal system 200 provides the following considerable benefits to patients, dental healthcare professionals and payers:
For patients, the test is objective, non-invasive, painless, bloodless and inexpensive.
For dentists, the test is fast, accurate, objective and digital. Dentists can immediately provide patients and payers with a paper or electronic copy of the test results and are expected to generate substantial additional practice revenues from additional periodontal testing and early-stage periodontal disease treatment fees. Proof that treatment is necessary can be shown to the patient on-screen and e-mailed to payers. A back-up copy of each patient's digital records may be stored in a HIPAA compliant manner at a remote Web-Site.
For payers, including third party payers, the test can accurately, objectively and digitally confirm the presence and extent of periodontal disease. More and more dental insurance companies and other payers are requiring digital proof of the patient's condition from dentists to confirm that treatment was necessary.

If periodontal disease is present, the dentist can quickly and accurately detect and diagnose the type and extent of the periodontal disease in its earliest stage, prescribe preventative treatment and perform on-going periodontal disease management to prevent its spread, the loss of diseased teeth 106 and the onset of other serious diseases and reduce healthcare costs. By using the digitized data and generating images taken every several months during the treatment period, both the dentist and the patient can readily confirm the treatment plan's effectiveness.

This type of preventative dentistry program results in better dental care for the patient. In fact, most dentists are expected to perform more examinations and treat those patients with mild periodontal disease that would otherwise have developed into more serious periodontal disease and then been referred to a periodontist for treatment. Those patients currently with more serious periodontal disease would still be referred to a periodontist.

The periodontal system 200 provides considerably more accurate and detailed information than the standard manual probe and analog method currently used by dentists and dental hygienists for periodontal tests, which requires the repeated, frequently painful insertion of a sharp metal probe into the crevice between the tooth and the gum. The manual probe and analog method is very inaccurate and can over- or under-estimate the patient's true condition by 1 mm or more. As a result, the ability of current manual probe method to diagnose periodontal disease in its very early stages is very difficult. Even the same dentist, or different dentists performing periodontal examinations on the same patient, can derive significantly different measurements. This happens for many reasons, including the probe not always being placed in the exact same location, the amount of pressure applied, the presence of granulation tissue due to infection, the skill and experience of the dentist or dental hygienist, patient movement, etc.

By contrast, the periodontal system's 200 dental imaging technology provides a significantly more accurate, consistent, reproducible measurement and diagnosis of periodontal disease and therefore earlier disease treatment opportunities because its margin for error is only +/−0.1 to 0.5 mm and the smallest changes can be easily and quickly recognized and treated. Preferably, the margin of error is 0.1-0.3 mm. More preferably, the margin of error is approximately 0.1 to 0.2 mm.

In addition to diagnosis, the periodontal system 200 may be used to monitor the progress of healing during treatment. Monitoring the progress of healing during treatment is possible because the system and method of the present invention is noninvasive and hence, does not tear or disrupt soft, healing tissue during use. This is in contrast to the conventional method of measuring periodontal disease, which requires insertion of a sharp probe between the tooth 106 and gum 102, which can result in tearing of the healing tissue.

In another embodiment of the invention, the periodontal system 200 may be used to assist in the treatment of periodontal disease. In this embodiment, medication is added to unit's liquid reservoir 236 or to the fluid from the hand piece 202. In still another embodiment, the system is able to detect the presence of calculus 110 on the tooth's 106 surface below the gum line 124 so it can be more easily and effectively removed. Further, the completeness of calculus 110 removal can be monitored by subsequent use of the system 200.

Another embodiment of the invention permits dentists or dental hygienists to determine how many measurements they want to be obtained on each tooth. This embodiment includes software that allows the handling of the large amount of digital data collected and stored. The software will enable dentists to obtain and store their patients' data on their office computers. In one aspect of this invention, the dentists or dental hygienists can operate the periodontal system 200 in continuous mode. In this mode, once triggered, the hand piece 202 automatically repeatedly emits pulses at regular intervals. The dental examiner sweeps the probe tip from one interdental space across the surface of the tooth to the adjacent interdental space. Preferably, the dental examiner performs a first continuous scan along the facial surface of the tooth and a second scan along the lingual surface of the tooth. In this manner, a profile of the bottom of the pocket can be generated rather than only gathering data from a few representative points. The total number of data points taken in this embodiment depends on the frequency of the transducer and the rate the dental examiner drags the hand piece 202 across the tooth 100, 106. Dozens, hundreds, even thousands of data points may be taken. In this manner, focal disease in the periodontal pocket 112 may be detected. In one aspect of this embodiment, all of the teeth 100, 106 may be scanned by the dentist or dental hygienist. In another aspect of this embodiment, only those teeth 106 that have previously identified as exhibiting periodontal disease are scanned in continuous mode, the remaining teeth 100 scanned with discretely triggered pulses. In still another aspect of this embodiment of the invention, geopositional technology may be used in combination with a fixed reference in the mouth to assist in defining the location and profile of the periodontal pocket 112.

Another embodiment of the invention can obtain complete and highly accurate readings and 3-D images of all of the patient's teeth and may be able to eliminate the need for dentists to obtain dental x-rays.

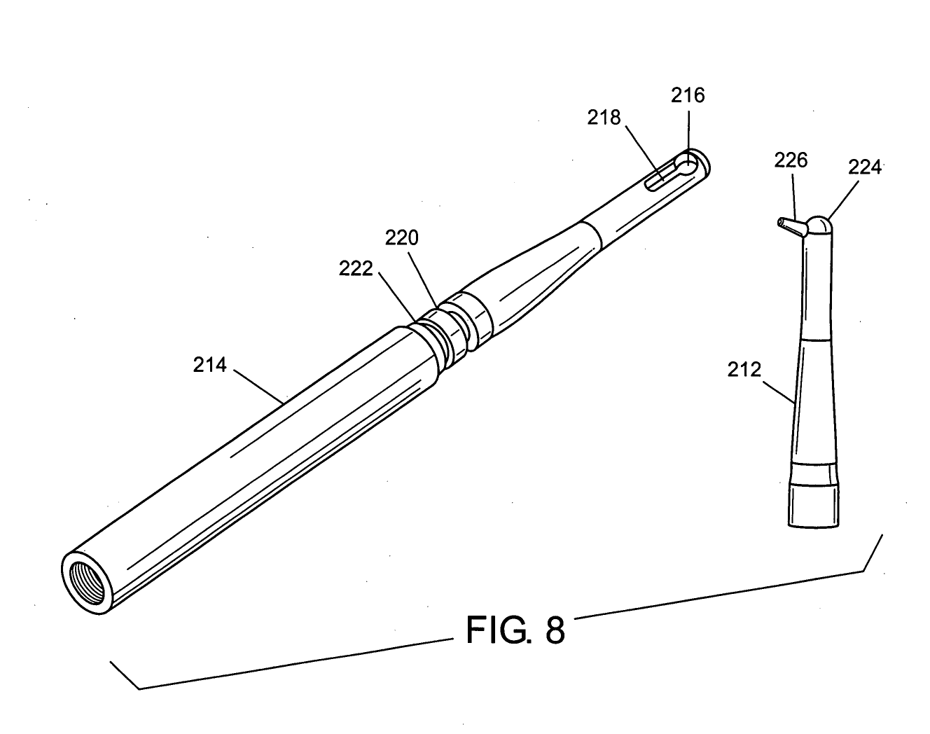

One embodiment of the invention is illustrated in FIGS. 8 and 9. This embodiment provides a hand piece 202 having a straight handle 214. One end of the handle 214 includes a cavity 216 adapted to hold a transducer (not shown). Adjacent to cavity 216 is an alignment slot 218. The alignment slot 218 mates with a protrusion in a disposable cover 212. The combination alignment slot 218 and the protrusion, greatly improve the reliability of alignment of the when placing a new disposable cover 212 on the handle 214. Located in a central portion of the handle 214, is a first circumferential slot 220. In one embodiment of the invention, the first circumferential slot 220 is provided with an helical spring (not shown). When a disposable cover 212 is pushed in place on the handle 214, the helical spring mates with a slot in the interior of the disposable cover 212, providing a snap fit. Also located in a central portion of the handle 214, is a second circumferential slot 222. Preferably, an O-ring is inserted in the second circumferential slot 222 to provide a seal. The disposable cover 212 includes a head portion 224 that covers the transducer and a probe tip 226 from which the ultrasonic waves are emitted. In one aspect of the invention, the disposable cover 212 may be provided with a safety feature that renders the disposable cover unusable after initial use. In another aspect, the disposable cover 212 includes an identification feature such as a serial number. The periodontal system 200 may be provided with a sensor to read the identification feature and determine if the disposable cover 212 has already been used. If the disposable cover 212 has already been used, the periodontal system 200 may refuse to allow further examination until a new disposable cover 212 is provided.

In one embodiment of the invention, the probe tip 226 is sized to fit snugly in the interdental space between teeth. As the location of this space does not vary, it provides a fixed reference point for taking periodontal measurements. The hand piece 202 is particularly advantageous because the probe tip 226 can be located behind the papilla. In this configuration, the hand piece 202 can be used to measure the deterioration of periodontal tissue (gum 102, periodontal ligament 103, 126, and the specula of bone between the teeth) due to periodontal disease.

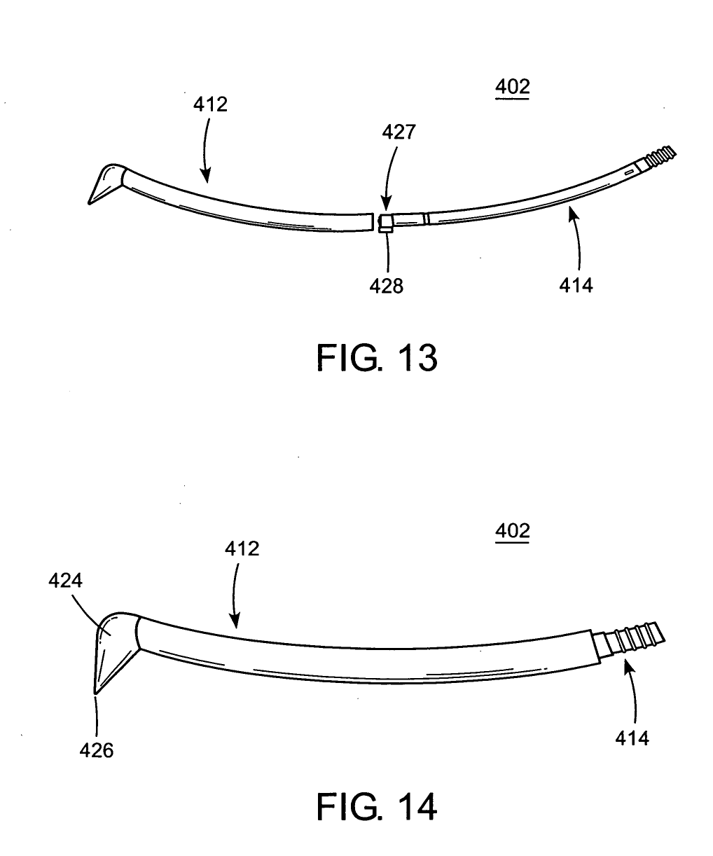

In an alternative embodiment of the invention, the periodontal system 200 includes hand piece 402, illustrated in FIGS. 13 and 14. The present inventors have recognized that the efficiency of the ultrasonic probe is significantly enhanced if the transducer 227 is located close to the probe tip 426. On the other hand, due to the concern of the spread of disease, it is necessary to sterilize that portion of the probe that enters the patient's mouth. The inventors have determined, unfortunately, that all current methods of sterilization, such as autoclaving and chemical washing, can damage the transducer 227, adversely affecting the useful life of the hand piece and its accuracy over time.

The inventors have discovered that the hand piece 402 of the present embodiment can be fitted with an easily removable cover 412. With this arrangement, the transducer 227 may be located in the head 424 of the hand piece 202, close to the probe tip 426. After a periodontal examination in one embodiment of the invention, the removable cover 412 can be removed and thrown away and a new removable cover 412 placed over the head 424. In still another embodiment of the invention, the removable cover 412 may be reused after sterilization. That is, the removable cover 412 may be removed from the hand piece 202, separately sterilized, and reattached to the handle 414. In another aspect of the invention, the hand piece 202 is connected to the display/controller unit 204 (FIG. 3) using quick-connect/disconnect electrical 238 and liquid flow connectors 240 that make it easy to quickly move the system between operatories.

FIG. 10 illustrates another embodiment of the invention. In this embodiment, the hand piece 202 includes an acoustic lens 228. The inventors have discovered that the efficiency of the hand piece 202 can be significantly increased by focusing the sound wave from the transducer 227 with an acoustic lens 228. Typically, the transducer 227 has an area much larger than the area of the exit opening of the probe tip 226 of the hand piece 202. Without an acoustic lens 228, much of the sound wave from the transducer 227 bounces off the inside walls of the probe head 224 as the probe head 224 narrows towards the tip 226. However, with an acoustic lens 228, the sound may be focused to the size of the exit opening of the probe tip 226.

In still another embodiment of the invention, the transducer 227 of the hand piece 202 is operated at intermediate frequencies. It is known that high frequency sound waves yield higher resolution, while low frequency sound waves have higher penetration. Typically, prior art ultrasonic devices have been designed to operate at frequencies of 2-5 MHz when high penetration was required and 15-20 MHz when higher resolution was required. In one aspect of the present invention, the inventors have discovered that a transducer 227 that uses frequencies between 5 and 15 MHz can yield both high resolution and high penetration. In one preferred embodiment of the invention, the transducer frequency is approximately 10 MHz.

Tip Shape Determination

Design Constraints

The shape of the tip 226 is preferably designed to ensure patient comfort and ease of use. It should also be compatible with the selected transducer, i.e. placing the focal point of the transducer in the region of interest.

Length

In one embodiment of the invention, the general length was determined to be approximately 10 mm to allow enough room for the medical professional to properly position the probe, but still provide enough length for the medical professional to visually determine the angular position. With this distance as a guide, an available transducer with an appropriate focal length (13.25 mm) was identified. The final length of the tip was then determined to place the focal point 244 approximately 2 mm beyond the end of the tip.

Diameter and Profile

In this embodiment, the inner diameter and profile of the tip 226 was determined from the beam diameter of the ultrasound pulse. The tip 226 surrounds but does not encroach upon the ultrasonic beam 242, ensuring that the pulse will not be reflected by the tip 226. Therefore, when properly aligned, the tip should not be visible in the ultrasound echoes. FIG. 34 illustrates the ultrasonic beam and the tip geometry.

Water Path Design

Design Constraints

The flow rate should be low enough to ensure patient comfort, but high enough to provide adequate acoustic coupling between the transducer and the patient.

Average Flow Rate

Theoretically, the water flow rate should have a negligible effect on the time of flight measurements of the ultrasound echoes. While the speed of the outgoing pulse is increased by the velocity of the water, the speed of the incoming echo is decreased by the same amount. However, turbulent flow could cause noise or distortions in the signal; therefore the velocity is preferably limited to ensure laminar flow (Reynolds Number<1000) through the tip. With a minimum tip diameter of 0.5 in, the maximum laminar velocity is approximately 3.1 inches per second:
v=(Re v)/d
where:
v is the velocity of the flow
Re is the Reynolds Number (1000)
v is the kinematic viscosity of water (1.01×10⁻⁵ m²/s)
d is the minimum inner diameter of the tip (0.5 in)

The maximum laminar flow rate is 604 mL per minute:
Q=vA
where:
q is the flow rate
v is the velocity of the flow (3.1 in/s)
A is the cross sectional area of the tip (0.196 in²)

Tests were also performed to verify the effects of flow rate on the measurements. This study gathered waveform data for reflections from a nominal 4.763 mm thick (4.752 mm measured) flat aluminum plate containing 1 mm diameter circular through hole that is placed on top of a second aluminum plate containing no holes. An immersion transducer (Xactex, 10 MHz, 13.25 mm focal length, 0.67 mm beam diameter) with an 11.055 mm tip was used. Data was collected at nine separate flow rates with the reflector positioned approximately 1 mm from the end of the tip. Flow rate was adjusted using a Harvard Apparatus PHD 2000 programmable syringe pump with a 10 mL Hamilton gas-tight syringe. For each flow rate, the three consecutive waveforms measurements were recorded.

The results of this study are shown in FIG. 35. Flow rate had little effect on the determination of the distance to the flat plate. However, the signal produced from the hole did show variation with flow, with a change in measurement between 5 and 7.5 mL/min. From this data, a target range of flow rates from 10 to 15 mL/min was selected. This range provides consistent measurements throughout the range and provides sufficient flow for acoustic coupling.

Flow Pulsations

Preferably, the periodontal system 200 uses a diaphragm pump with a running speed of approximately 30 Hz. This equates to a cycle period of 30 ms. Preferably, the data acquisition time of an entire scan is 30 us, or 1/1000 of a pump cycle. Therefore, even though the diaphragm produces observable pulsations in flow rate, the flow can reasonably be assumed to be stable during the duration of the scan acquisition.

Air Bubble Elimination

Air bubbles, including microbubbles, dramatically increase the attenuation of the signal and reduce signal strength. The flow path is preferably designed to facilitate the flushing of air bubbles out of the system. Areas in the flow path where air could get trapped (i.e. local high points) are preferably minimized. Additionally, the water is preferably deaerated upstream of the hand piece. This is preferably accomplished by pulling a vacuum on one side of a PTFE filter. The surface tension of water prevents liquid from flowing through this filter, but air and other gases can flow freely through it.

A preferred embodiment of the invention provides a completely noninvasive method of measuring the gum line 124 (FIG. 2) to the cemento-enamel junction 132, and the determination of the depth of the periodontal pocket 112 extending from the cemento-enamel junction 132 to the bottom of the periodontal pocket 112. In this embodiment, a sound wave is transmitted along the tooth 106 starting from the gum line 124. Returning echoes are analyzed by the discrimination analysis algorithm. Echoes from the cemento-enamel junction 132 fix its location relative to the gum line 124, while echoes from the bottom of the periodontal pocket 112 fix the location of the bottom of the periodontal pocket 112 relative to the gum line 124. The depth of the periodontal pocket is determined by subtracting the distance from the gum line 124 to the cemento-enamel junction 132 from the distance from the gum line 124 to the bottom of the pocket 112. Prior art ultrasonic periodontal devices, in contrast, either used an invasive probe to determine the location of the cemento-enamel junction 132 or measured the periodontal pocket 112 through the gum line 124, completely ignoring the cemento-enamel junction 132.

In one aspect of the invention, the return pulses are amplified and transformed to separate peaks from noise. In one preferred embodiment of the invention, a wavelet algorithm is used in the transformation process. In still another embodiment of the invention, a discrimination analysis algorithm is used to aid in determining the identification of the various peaks. In still another embodiment, both a wavelet and a discrimination analysis algorithm are used.

In one embodiment, the display/controller unit 204 (FIG. 3) will provide a series of audible tones and/or visual signals to guide the dentist through the test thereby permitting the dentist to advance to the next tooth 100, 106 or to reverse back to the last tooth and re-test it if the image was not properly captured. These signals can also alert the dentist to the presence of unusually deep periodontal pockets 112 that may signify significant periodontal disease or other conditions that require attention or treatment.

The display/controller unit 204 is fully self-contained and will provide the necessary images on its own screen even if the signal cannot be transmitted outside of the room. In addition, all of the patient's information can be sent to the dentist's office computer wirelessly or via a cable connection so that patient information does not have to be re-entered.

In one embodiment of the invention, the hand piece 202 includes a disposable cover 212. The disposable hand piece cover 212 will be contained inside of a sterile, tamper-resistant package that also contains a disposable stylus that can be used on the system's touch screen for data entry purposes, an alcohol soaked gauze pad in a sterile pouch to wipe off the hand piece between patient tests and a see-through disposable plastic cover for the touch screen in the event of splatters. The package, all disposables and technology (including all enhancements) may be provided to dentists without charge in consideration of their paying a test fee.

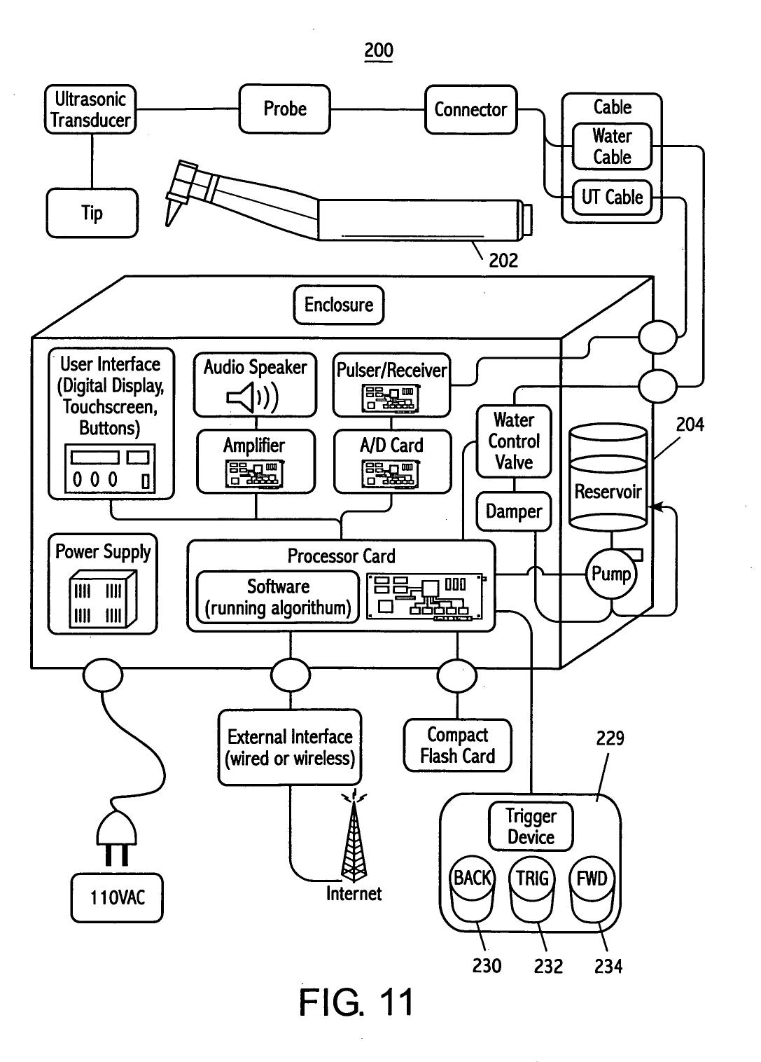

FIG. 11 illustrates still another embodiment of the invention. This embodiment of periodontal system 200 includes a hand piece 202 and a display/controller 204. This embodiment further includes a triggering device 229. In this embodiment, the triggering device 229 includes three foot pedals 230, 232, 234. The triggering device 229 activates the transducer 227 (FIG. 10) and initiates fluid flow. In another embodiment, the triggering device 229 is on the hand piece 204. In still another embodiment the triggering device 229 includes software that allows voice activation.

The following summarizes and describes various features of the software of the periodontal system 200 (FIG. 11). This summary describes how the software:
Controls the device's electronic components,
Interfaces with the end-user,
Executes the data acquisition algorithm, including initiation and receipt of the acoustic signal and the subsequent calculation of periodontal pocket depth,
Displays collected data,
Stores and protects patient information,
Calibrates the device, and
Communicates with external devices.

The periodontal system 200 is an ultrasonic probe system used in the measurement of a patient's periodontal condition. The periodontal system 200 consists of a handheld probe 202, a triggering mechanism and a compact display/controller unit 204. The probe 202 transmits an ultrasonic pulse into the periodontal pocket 112 of the patient through a stream of water, or other liquid (typically, required for acoustic coupling) and captures the echoes resulting from collision of the ultrasonic wave with anatomical features in the periodontal pocket 112. Embedded software running within the display/controller unit 204 uses an analysis algorithm to correlate the acoustic echo with the depth of the outer boundary of the periodontal ligament 130 (e.g. pocket depth in millimeters).

The software application is preferably supported by an embedded operating system running on the display/controller unit 204. The software application controls the periodontal system 200. Control features include:
Sending a signal to external hardware to emit an ultrasonic pulse upon receipt of a trigger signal from an external trigger mechanism (e.g., a foot pedals 230, 232, 234),
Controlling water flow to the handheld probe 202,
Acquiring the ultrasonic echo as a digitized electrical signal,
Performing the pocket depth calculation,
Calibrating the periodontal system 200, as necessary, between scans,
Providing audible prompts to the user denoting the end of a scan and readiness to perform a new scan,
Driving a touch screen and LCD display 205,
Reacting to user input via the triggering mechanism,
Storing data relating to the patient and all exams performed in a patient database,
Is capable of driving an external video display unit,
Is capable of receiving software upgrades,
Supporting network communications to transmit and receive patient data,
Communicating to a remote Internet portal.

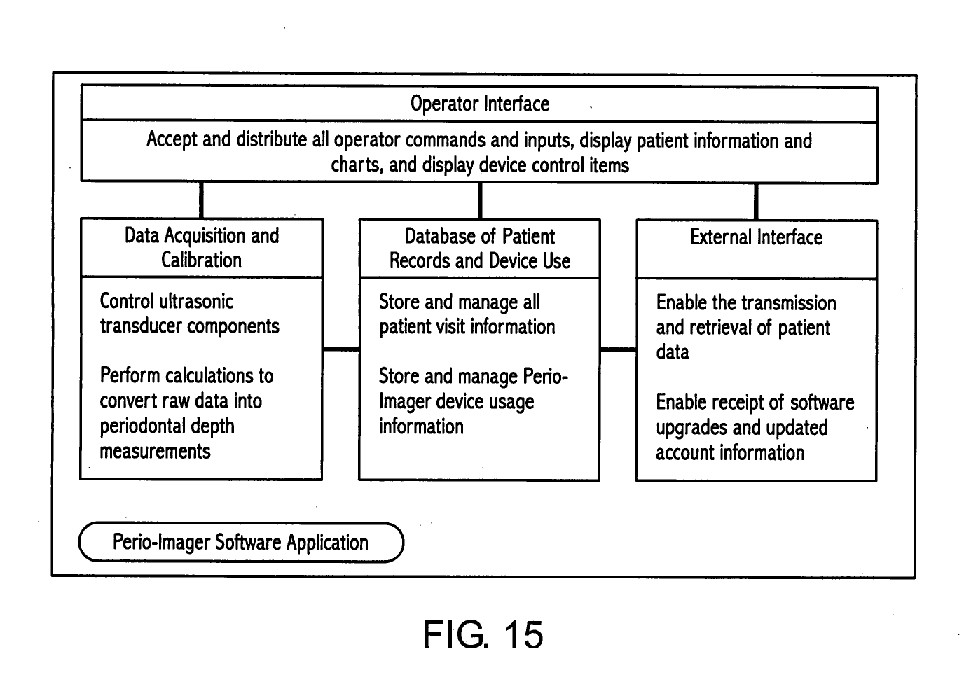

FIG. 15 illustrates an embodiment of the functional layout of the periodontal system 200. Table 2 contains a list of the major components and external systems that interface with the periodontal system's 200 software application, and indicates their main function in the device.

TABLE 2

| Component | Description |
|---|---|
| Ultrasonic Transducer | Emits and receives ultrasonic signals |
| Solenoid Valve Relay | Opens and closes water suply |
| Trigger Device | A trigger device accepts operator commands to |
| | 1) Sequence forward and backward to reach the desired |
| | tooth location for a given scan, and |
| | 2) Acquire a pocket depth measurement |
| Pulser/Receiver | Sends an electrical pulse to the transducer and receives the |
| | returning electrical signal created by the ultrasonic echo |
| Analog-to-Digital Converter | Samples the signal received by the pulser/receiver and |
| (A/D board) | converts samples to digital values |
| Single Board Computer (SBC) | Supports the operating system and runs the software |
| | application. Executes the data analysis algorithm and control |
| | commands and/or signals |
| Flat Panel Display | Video screen that is the main user interface medium |
| Touch-Sensitive Display | Accepts user input through a touch-sensitive screen integrated |
| (Touch Screen) | into the device flat panel display |
| Audio Speaker & Amplifier | Emits audio feedback to the user concerning data acquisition |
| | events and the state of device readiness |
| Compact Flash Memory | Provides data storage capacity |
| External Network Connection | Allows the user, via the software application, to upload or |
| | download patient visit data to or from a central data |
| | repository, and/or from device to device, and to download |
| | software upgrades. |

Software Environment
Operating System

In one preferred embodiment of the invention, the periodontal system 200 includes a microprocessor that runs using the Windows XP Embedded (XPe) Operating System, which is a componentized form of the Windows XP Professional Operating System. The componentization enables the operating system of the periodontal system 200 to be customized to include only those features of Windows XP necessary to the operation of the periodontal system 200, and the exclusion of those that are not.

Software Development Tools

The application is preferably an object-oriented Windows application written in the C++ coding language using Microsoft's Visual Studio 6.0 IDE (integrated development environment). The application may, however, be implemented using other computer languages and with other tools. Software modules including graphics tools, device driver programs for the A/D card, the touch screen control electronics, the trigger device, and the audio speaker are preferably included in the application or are accessed by the application via dynamically linked library (DLL) files.

Hardware Environment

Single Board Computer and Processor

The periodontal system's 200 software application preferably runs on a single-board computer that supports and contains all of the interface hardware and software components. This computer preferably has a 1-GHz VIA Eden™ ESP 10000 processor with a VIA Technologies, Inc. Twister-T chipset (VT8606 and VT82C686B chips). Further, it preferably has 256 MB of RAM, connections for a keyboard and mouse, cathode-ray tube (CRT) and liquid crystal display (LCD) video interface connections, four universal serial bus (USB) ports, two Ethernet ports, one parallel/floppy port, one General Purpose Input/Output (GPIO) port, and four serial ports. It has PC/104 and PC/104+ interfaces, and a Compact Flash adapter. Other combinations input and output connections are also possible and within the scope of the invention.

Touchscreen Controller

A 4-wire resistive touch-sensitive touchscreen, mounted in front of an LCD is the preferred way for operator interaction with the periodontal system's 200 software. The touch screen is preferably used in the same manner as a one-button mouse. A controller board preferably converts the analog signals coming from the touchscreen into X and Y coordinates and selection events, and communicates this data over a USB interface to the computer. Driver software is typically required for the controller board to operate. This driver application preferably includes touchscreen calibration software that initially correlates LCD X and Y coordinates with touchscreen X and Y coordinates to account for misalignment between the two reference frames. In one embodiment, the driver application is not part of the periodontal system's 200 software application, but is used by the operating system to allow it to receive and use the mouse-like inputs coming from the controller board. The touchscreen is preferably calibrated before the device is delivered to the user. Under normal circumstances, the user will not calibrate the touchscreen.

Trigger Device

The periodontal system 200 is preferably controlled by the operator during patient examinations by a trigger device. The trigger device sends commands to the periodontal system 200 to begin an acquisition or to move to the next tooth location. Commands given by the trigger device are preferably mapped to unique keyboard sequences, meaning the operating system interprets each type of command received from the trigger device as a certain keyboard sequence. The periodontal system's 200 software application waits for these keyboard sequences (generated by the trigger device), and takes specific actions in response to each reported sequence. The keyboard-trigger device mapping is shown below in Table 3.

The trigger device for the rapid prototype and investigational periodontal system 200 is preferably a three-position foot pedal that is connected to the periodontal system's 200 microprocessor through a USB interface. The Savant USB driver for Windows, for example, may be used to accommodate the USB communication between the operating system and the foot pedal. The trigger device can also be activated using buttons in the hand piece or using voice recognition software.

TABLE 3

| Trigger Device | Keyboard Sequence | Function During Examinations |
|---|---|---|
| Left Button | ALT + 1 | Move to previous tooth location |
| Middle Button | ALT + 2 | Perform periodontal depth |
| | | acquisition |
| Right Button | ALT + 3 | Move to next tooth location |

Input/Output

Preferably, the software application interfaces with three components, a water control solenoid valve, the Pulser/Receiver and the A/D card via the display/controller's 204 parallel port. Preferably, all three are triggered when they receive a digital HIGH signal from the parallel port channel to which they are connected. Typically, the parallel port is commanded to send these signals when the data acquisition software receives an appropriate command from the operator interface. A parallel port software module may be written into the periodontal system's 200 software application and implement the functions necessary to configure and use this interface. The pulser/receiver and A/D card may be activated by the same parallel port channel.

When the solenoid valve opens, water preferably passes through the valve to the periodontal system's 200 hand piece 202. The Pulser/Receiver preferably sends a negative voltage pulse to the ultrasonic transducer 227, which converts that pulse into acoustic energy. The transducer 227 then receives and converts the acoustic echo returning to it back into an electrical signal, which is then sent back to the Pulser/Receiver, and sampled by the A/D board.

Analog/Digital Board

Preferably, the analog/digital (A/D) board communicates over the PCI bus on the periodontal system's 200 microprocessor. The periodontal system's 200 software application preferably includes a software module that contains all of the driver functions and variables necessary to initialize, trigger, and retrieve data from this board. The functions included in this module are preferably supplied by the manufacturer of the board. In one aspect of the invention, 12-bit digital samples of the returning echo waveform are acquired at a rate of 100 megasamples per second. In other aspects, the digital samples may comprise more or less than 12 bits. In still other aspects, the sample rate may be more or less than 100 megasamples per second.

Flat Panel Display

The transflective TFT LCD flat-panel display preferably does not require any additional software or drivers to operate, and is controlled by drivers resident in the operating system. Preferably, the BIOS is configured to support both an LCD and an external CRT monitor. The LCD connection is preferably internal to the periodontal system 200, while a connection to an external CRT may be provided at the rear of the device.

Audio Speaker

The audio speaker is preferably supported by drivers resident in the operating system. Software commands to play selected audio files (e.g., .WAV) are preferably issued using platform (Windows XP Embedded) functions.

Compact Flash

Preferably, the compact flash card functions as the storage medium of the periodontal system's 200. Preferably, it contains the operating system (XPe), the periodontal system 200 software application, and a database of patient records. The operating system is configured to boot from the compact flash card. Preferably, the compact flash card is type II, 1 GB in size, and formatted as fixed media. Any suitable type and size, however, may be used.

External Network Interface

Preferably, the periodontal system 200 can communicate to external devices through wired (Ethernet) or wireless (802.11x) connections. Preferably, the Ethernet hardware is integrated into the computer and drivers are supplied by the manufacturer. The wireless hardware is an optional module that may be added to the computer. The make and model of the wireless Ethernet module are not critical to the invention.

FIG. 15 provides a graphical summary of the software application's four main tasks. These include the operator interface, data acquisition and calibration, the maintenance of a database of patient information and data, and external interface.

The operator interface forms the backbone of the application, and all other functions of the application are preferably controlled from commands received through this interface. The operator may enter commands or data through a touch-sensitive screen and trigger device, and receive information back via a series of interface screens. The operator may also be given audio feedback via the audio speaker.

Software elements that control the acquisition and analysis of data typically receive their instructions from the operator interface, and then execute the necessary software and hardware procedures to perform those tasks. Similarly, these elements may also control the task of calibration of the Data Acquisition system.

Preferably, patient data is stored in a database created and maintained by the application. This application preferably controls the entry, modification and protection of patient data.

External interface software preferable to communicate over USB and Ethernet ports with external devices such as the trigger device or an external monitor. Preferably, the external interface creates the ability to communicate with a web-based repository where periodontal data can be stored or retrieved. Finally, it preferably allows the passing of patient data and information from one periodontal system 200 device to another within the dentist's office, and to and from external computers over a secure line of communication.

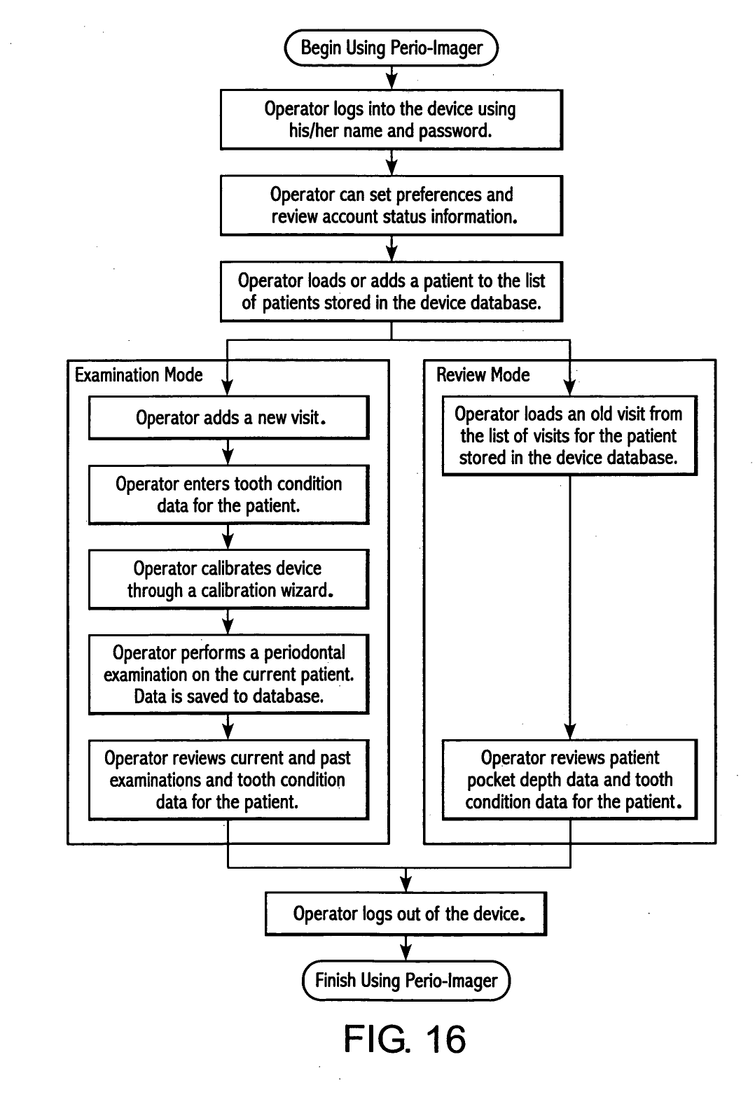

The typical process of performing an examination is shown in FIG. 16. An operator typically logs into the device through a login screen. The operator can then load an existing patient from the patient database or create a new one that is then stored in the patient database. At this point, if the operator creates a new visit they are functioning in Examination Mode and can perform an examination and enter in tooth condition data. Usage of this mode is preferably considered a usage towards business accounting purposes. Preferably, if the operator simply views an existing patient visit they are functioning in Review Mode and can view patient data and standard reports on the patients periodontal and tooth condition data.

Graphical User Interface Screens

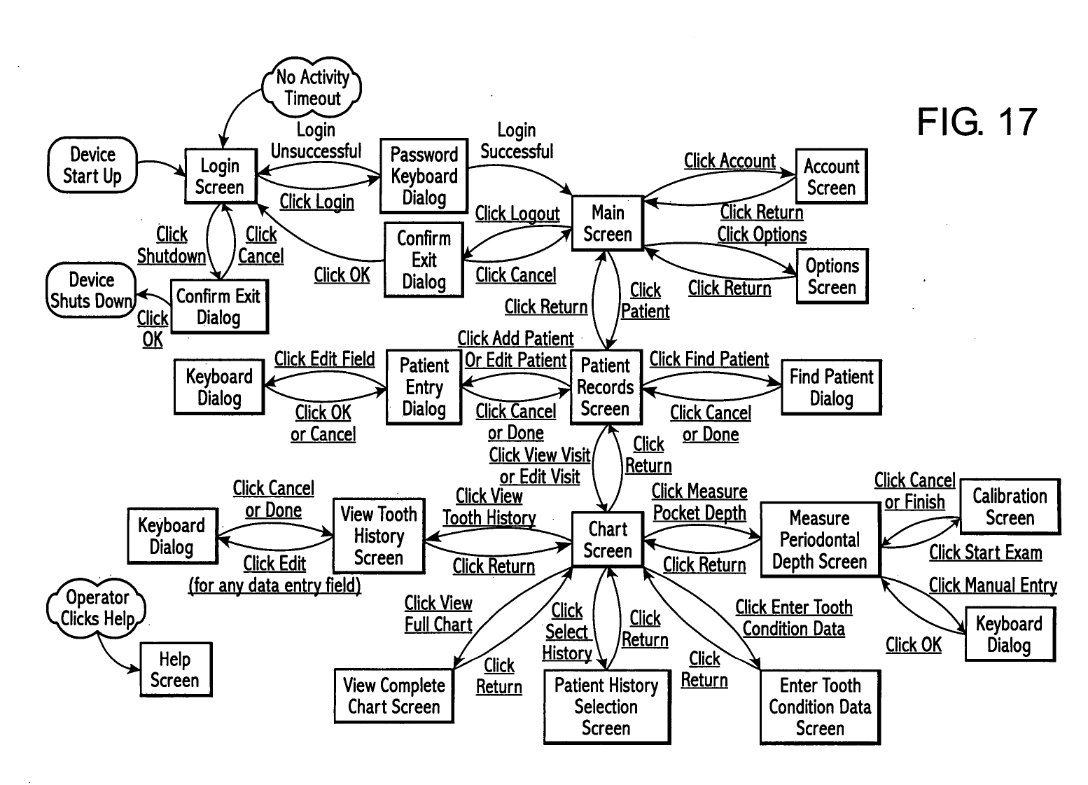

The operator interface consists of several screens with specific functions as shown in FIG. 17. A commercial software library may be used to graphically enhance the visual appearance of the dialog boxes and operator interface screens.

The entryway into the application is preferably through the Login Screen, which allows operators to login using a password. Once logged in, the operator is at the Main Screen where they can view account information through the Account Screen, setup preferences in the Operator Screen, or review patient information through the Patient Records Screen. This screen allows the operator to select current patients to load or to create new patients.

Once a patient is loaded the operator can view or edit the record through the Chart Screen. The chart screen shows information about the patient including pocket depth and tooth condition data including any overlays selected through the Patient History Selection Screen. The operator can edit tooth condition data for the patient through the Enter Tooth Condition Data Screen, or can perform a periodontal examination through the Measure Pocket Depth Screen. The operator can view reports on the patient through the View Complete Chart Screen and the View Tooth History Screen.

Common Screen Information

The Chart Screen, Enter Tooth Condition Data Screen, Measure Pocket Depth Screen, View Complete Chart Screen, View Tooth History Screen preferably have the following fields at the top and bottom of the screen:
Operator name text.
Patient name text.
The date and time of the currently loaded visit, which is the current date and time if it is a new visit.
Status text, which is used to prompt the operator with status information relative to events on the screen.
Current date and time, updated once per minute in the status bar.
Help button that takes the operator to the Help Screen and loads the relevant section in the help topics index for the screen.
   Tooth Condition Tables

Preferably, several screens contain charts with tooth condition data presented in a tabular form. In one embodiment, due to size constraints and in order to maximize readability, the chart presents data from only eight teeth at a time (one quadrant of the mouth). In other embodiments of the invention, any number of teeth may be presented up to and including all of the teeth. For example, the dentist may choose to view a single tooth with periodontal disease, or one or two teeth on each side of the diseased tooth (a total of three or five teeth, respectively).

Certain data is preferably displayed for each location on a tooth. The locations may be denoted as:
D for the location on the Distal side of the tooth.
M for the location on the Mesial side of the tooth.
C for the Central location on the tooth (i.e. between the Mesial and Distal location).
   In other aspects of the invention data may taken in the interdental space and denoted as I.

Preferably, the data displayed for each of the three locations on a side of a tooth (and in the interdental space) is:
Pocket Depth (0.0-9.9)
Furcation (0-3)
Recession (0-3, or using the Miller Classification which is determined in the Options screen).

The tooth condition data displayed for each tooth may include:
Suppuration (S for yes, blank for no)
Mobility (0-3)
Edema (0-3)
Missing (Missing for yes, blank for no).
   Login Screen

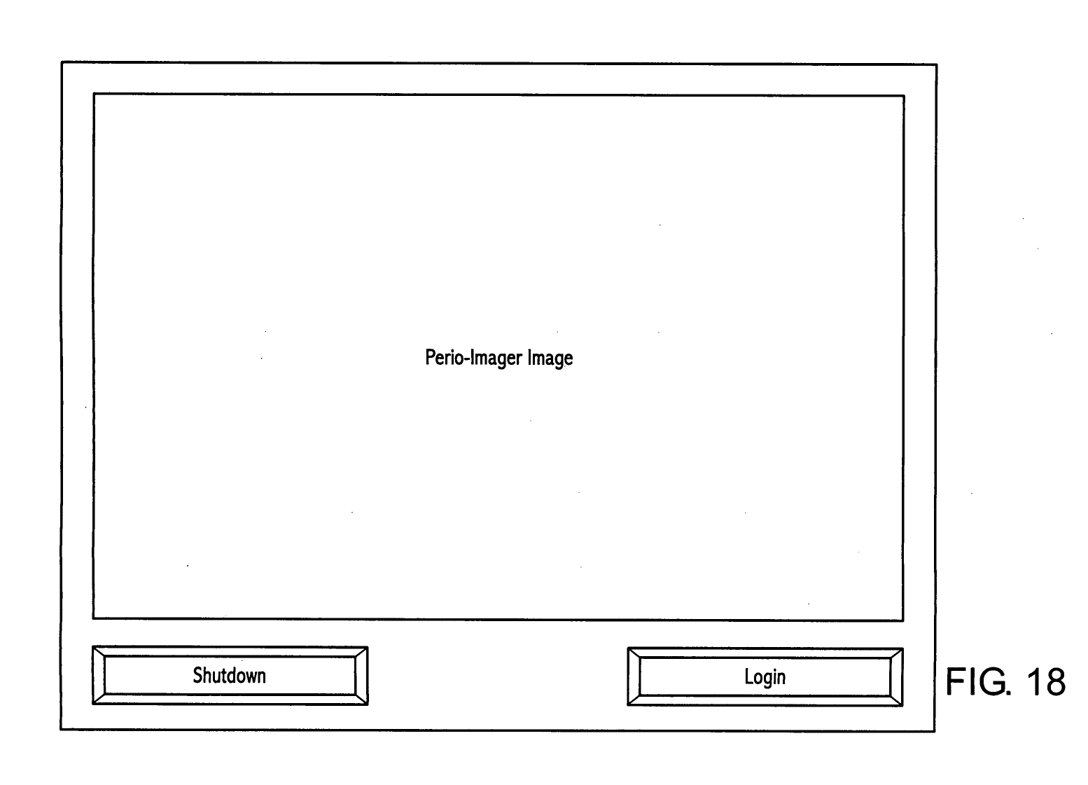

This screen is preferably the initial screen displayed when the device is powered on. Preferably, when this screen is present no other screens or functions of the device can be activated except to login to the device or to shutdown (power down) the device. When the Login button is clicked the operator can login and begin using the device. This way, any data taken is associated with that operator. Preferably, when the operator is finished with any examination they should log off of the device from the Main Screen. When that occurs this screen preferably becomes the only screen visible. In one aspect of the invention, if the device is inactive for more than a configurable amount of time, this screen becomes active again. The login provides a layer of security for patients' medical records. FIG. 18 shows a notional view of the Login Screen.

The operator encounters the following fields and buttons in the Login Screen:
A. Login Button: This button opens a keyboard dialog that allows a person to login to the device. A person can login only as one of the selectable operators and with that operator's password. There is always a login available for “Administrator”, and when that person is logged on they have extra functions available to them (e.g. they can choose to add new operators in the Account screen).
B. Shutdown Button: This button allows the operator to “soft” shutdown the system. This is the preferred manner to shutdown the periodontal system 200 device.
C. Periodontal system 200 Image: This image is available to show the device and the PII logo.
   Main Screen

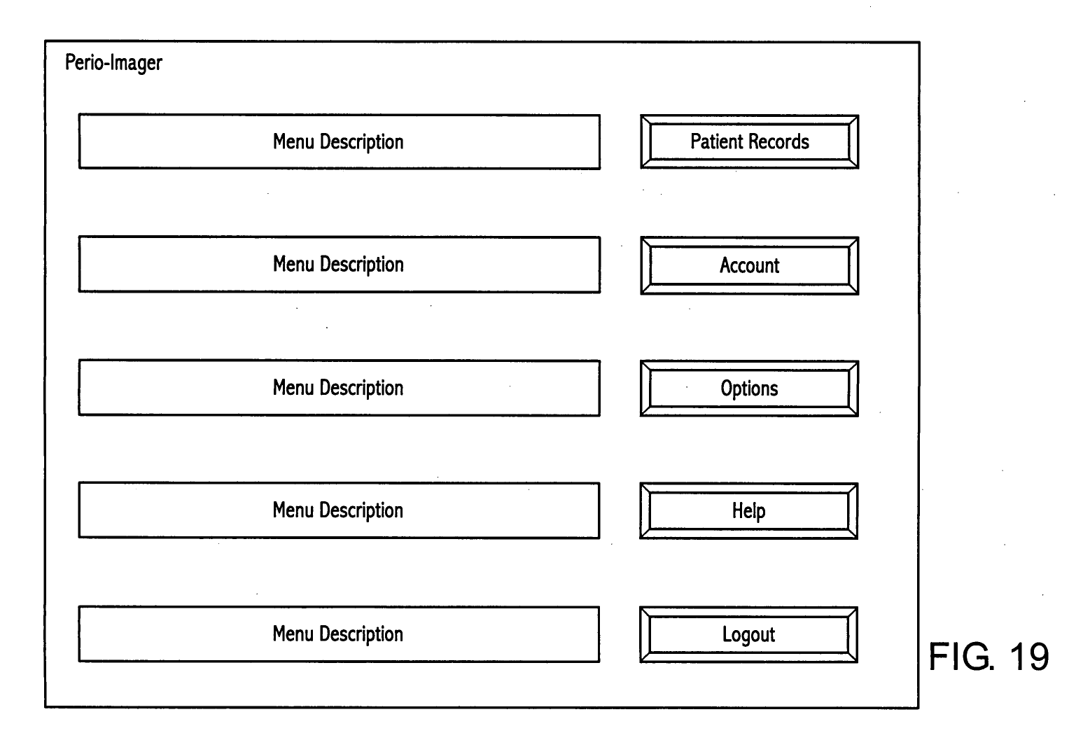

The Main Screen is preferably used as a gateway to the functions of the periodontal system 200 device. On this screen the operator can also logout or shutdown the device. FIG. 19 shows a notional view of the Main Screen. The Main Screen can also be used to promote products and services and provide dentist with access to a database containing the standard of care and recommended course of treatment applicable to the patient's periodontal condition.

The operator encounters the following fields and options in the Main Screen:
A. Menu Description: Textual information on the menu option
B. Menu Buttons: These buttons take the operator to various screens (Patient Records, Account, Options, Help, Login) or allow the current operator to logout.
   Account Screen

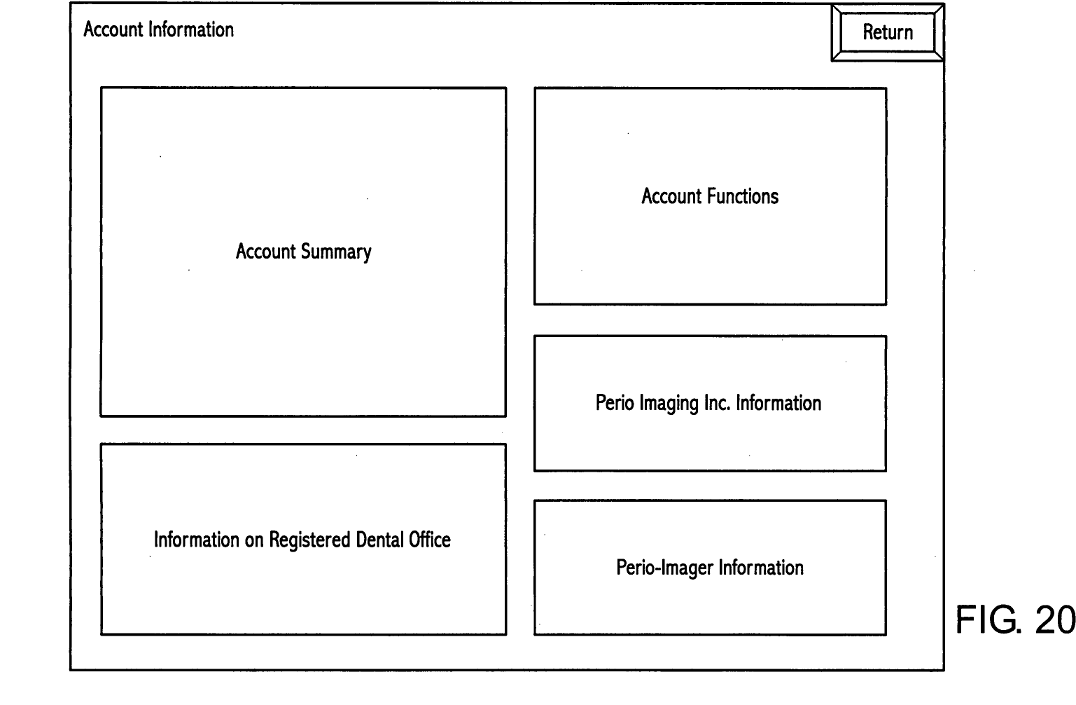

The Account Screen is used to display information about the system's manufacturer, to provide statistics about the usage of the device, and account information. A user will go to this screen to get information on current account status and an explanation on how to update their account. FIG. 20 shows a notional view of the Account Screen.

The operator encounters the following fields and options in the Account Screen:
A. Account Summary: This section preferably lists information on the dentist's account such as account status and the number of uses left on the device. If the number of permitted uses left in the account is low, the number displayed may be red in color and warning text appear below the number. If the number of permitted uses left is not low then the color of the number is preferably the normal text color.
B. Account Functions: This region preferably contains buttons that the “Administrator” operator can use perform account operations such as:
   Add a new operator
   Order more supplies from The Supplier
   Order more device usages from The Supplier
C. Information on Registered Dental Office: Textual display of the current owner of the device such as registration number and dentist office name and address.
D. Perio Imaging Inc. Information: Contains textual information on how to contact PII.
E. Periodontal system 200 Information: Shows the current device's serial number, as well as software and hardware version.
F. Return Button: This button takes the operator back to the Main Screen.
   Options Screen

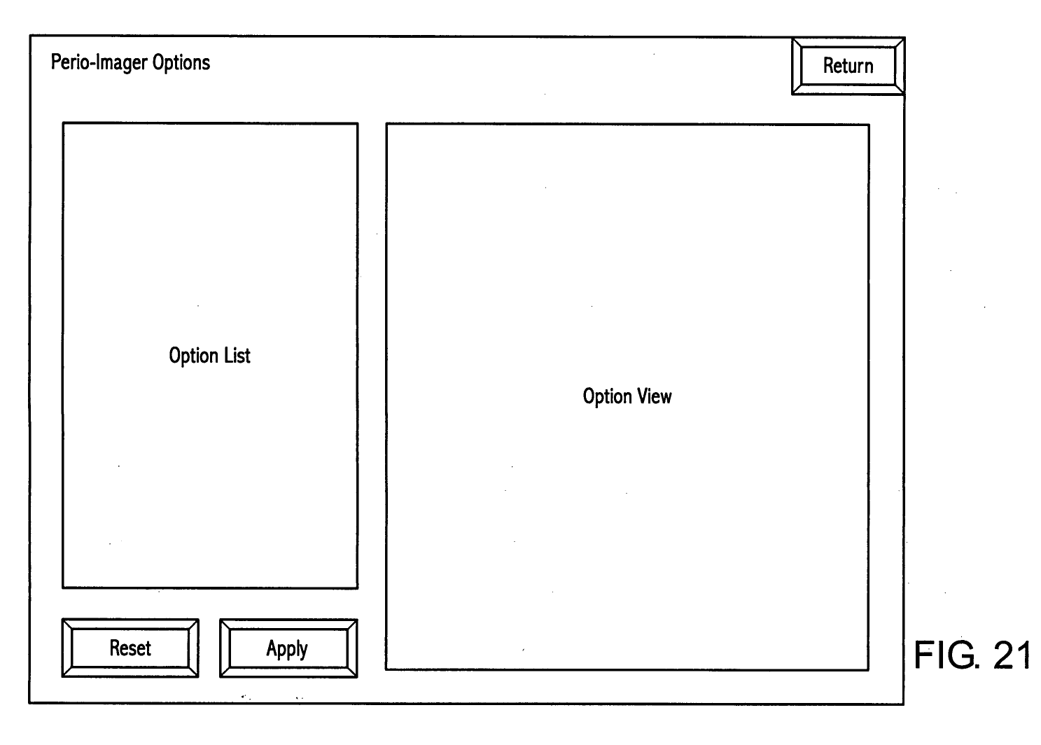

The Options Screen preferably allows the operator to set up and store his or her preferences for the device and for the process of measurement. Each option can be selected from a list. Once an option is selected, its specific controls are preferably displayed on the screen. FIG. 21 shows a notional view of the Options Screen.

Options that can be specified through the screen include:
Preference options for tooth measurement scan order
Speaker and microphone volume settings
Login screen timeout
Quick notes definitions.

The operator encounters the following fields and buttons in the Options Screen:
A. Option List: This presents the operator with a tree view of options to select for modification. Once selected, the controls for those options preferably become visible in the Option View.
B. Option View: The View area preferably houses all controls associated with the option set selected in the Option List.
C. Reset: This button preferably resets all options back to the defaults.
D. Apply: This button preferably applies all modified options to the device.
E. Return Button: This button preferably takes the operator back to the Main Screen.
   Patient Records Screen

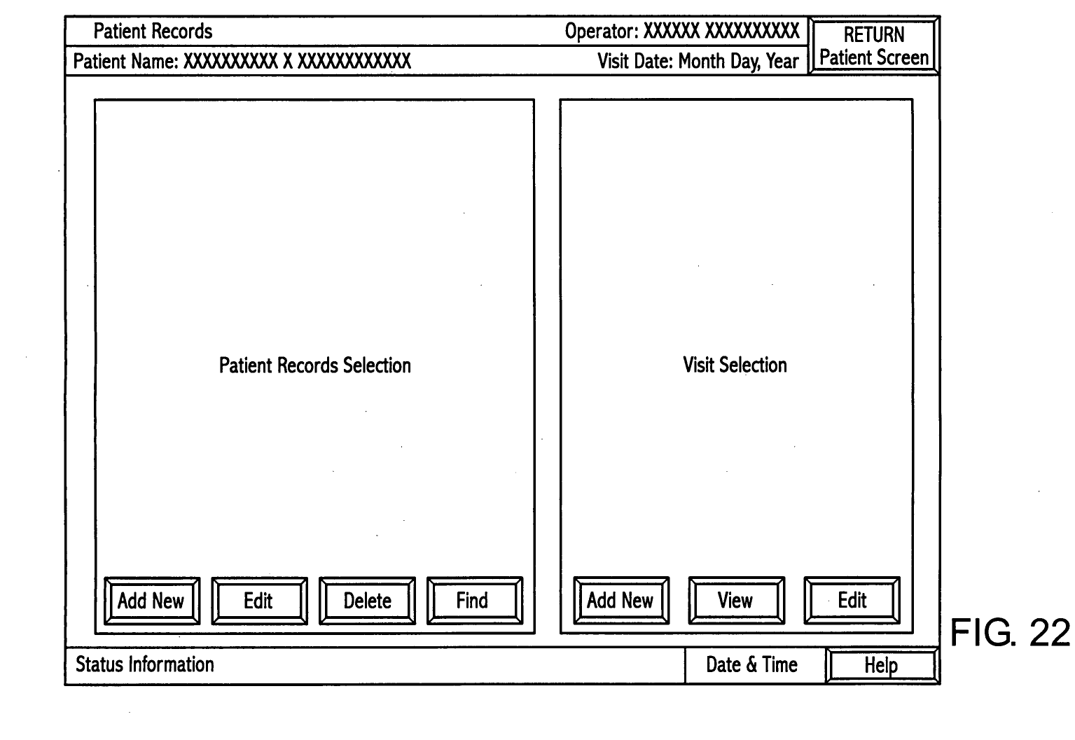

The Patient Records Screen allows the operator to add and edit patient records stored in the internal database. Each patient is preferably assigned a unique identification number (ID) that is stored internal to the device and is used when transferring data to external devices (such as the PII Internet Portal). FIG. 22 shows a notional view of the Patient Records Screen.

The operator encounters the following fields and options in the Patient Records Screen:
A. Patient Record Selection: This list preferably allows the operator to select a patient and a particular record. When the operator changes the patient selection the Visit Selection field preferably updates with the list of all past visits for that patient. In addition, the patient name, displayed at the top of the screen, is preferably updated. This field preferably has several functions:
   a. Add New: This button preferably opens a series of Keyboard dialogs that allows the entry of a new patient including (patient name, dentist office patient ID, date of birth, gender, and address). After entry the new patient name is preferably displayed in the Patient Record Selection field and is highlighted.
   b. Edit: This button preferably opens a series of Keyboard dialogs that allows the operator to edit the patient information (patient name, dentist office patient ID, date of birth, gender, and address).
   c. Delete: This button preferably brings up a confirmation dialog to confirm if the operator wants to delete the patient. Note: a patient can only be deleted if that patient does not have any visits.
   d. Find: This buttons preferably opens a dialog that allows the operator to enter the first few letters of the patients last name to allow for quickly finding a patient in the Patient Record Selection list.
B. Visit Selection: This list preferably allows the operator to view all of the visits that have been performed for the currently selected patient. If the patient is newly created and there are no visits, this list is preferably empty. When a visit is selected, the visit date, displayed at the top of the screen, is preferably updated. This field preferably has several functions:
   a. Add New: This button preferably allows the operator to create a new visit record for the currently selected patient. A new entry in the Visit Record Selection list for the current patient is preferably created, displayed, and selected. The new entry preferably references the current time and date. A warning dialog is preferably displayed that asks the operator to confirm that they want to proceed with adding a new visit.
   b. View: This button takes the Operator to the Chart Screen in Review mode (see FIG. 16). This button is preferably enabled only after a visit has been selected in the Visit Selection list, or after a new visit has been added.
   c. Edit: This button preferably takes the Operator to the Chart Screen in Examination mode (see FIG. 16). This button is preferably enabled only after a visit has been selected in the Visit Selection list, or after a new visit has been added.
C. Return Button: This button preferably takes the operator back to the Main Screen.
   Patient Chart Screen

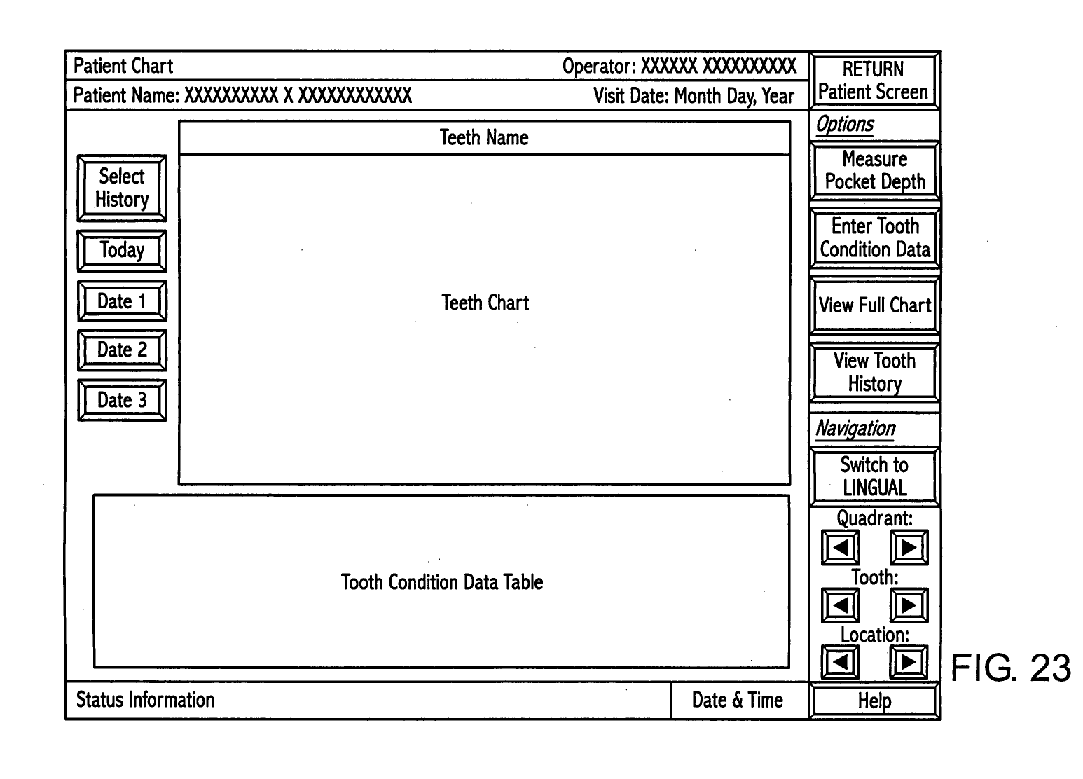

The Patient Chart Screen is used to display all the periodontal measurement data from a patient visit overlaid on images of teeth and tabular presentation of tooth condition data. In addition, the operator can choose to display patient data from past visits. All data displayed is retrieved from the internal database. FIG. 23 shows a notional view of the Patient Chart Screen.

The operator encounters the following fields and options in the Patient Chart Screen:
A. Teeth Chart: Periodontal data is preferably presented as points for each measurement and lines between the points overlaid on an image of teeth. The specific data displayed is preferably controlled by the history selections and the Tooth Selection Arrows. Preferably, a region of eight teeth is highlighted and the data for these teeth is tabulated in the Tooth Condition Data Table.
B. Teeth Name: Textual name of the currently selected tooth.
C. Tooth Condition Data Table: Tabulated tooth condition data for the currently selected quadrant according to Tooth Condition Tables.
D. Option Buttons:
   a. Measure Pocket Depth: This button preferably takes the operator to the Measure Pocket Depth Screen. Preferably this button is only enabled when the operator is in Examination Mode.
   b. Enter Tooth Condition Data: This button preferably takes the operator to the Enter Tooth Condition Data Screen. Preferably this button is only enabled when the operator is in Examination Mode.
   c. View Full Chart: This button preferably takes the operator to the View Full Chart Screen.
   d. View Tooth History: This button preferably takes the operator to the View Tooth History Screen.
E. Navigation buttons:
   a. Switch to Facial/Lingual Button: These buttons preferably select between facial and lingual data to be presented on the Teeth Chart and the Tooth Condition Data Table.
   b. Tooth Selection Arrows: The operator can use these buttons to cycle which set of teeth is being highlighted in the Teeth Chart and therefore also tabulated in the Tooth Condition Data Table. The operator can cycle by location on tooth, by tooth, or by quadrant.
F. Select History Button: This button preferably takes the operator to the Patient History Selection Screen. It allows the operator to select which past examinations to overlay on the Teeth Chart, and which past examinations to display on the View Tooth History Screen.
G. Today & Dates 1-3 Buttons: These buttons preferably control which overlay is currently highlighted on the Teeth Chart. The histories are selected through the Select History button. Dates 1-3 are appropriately disabled if there are less than three history selections.
H. Return Button: This button takes the operator back to the Patient Records Screen.
   Enter Tooth Condition Data Screen

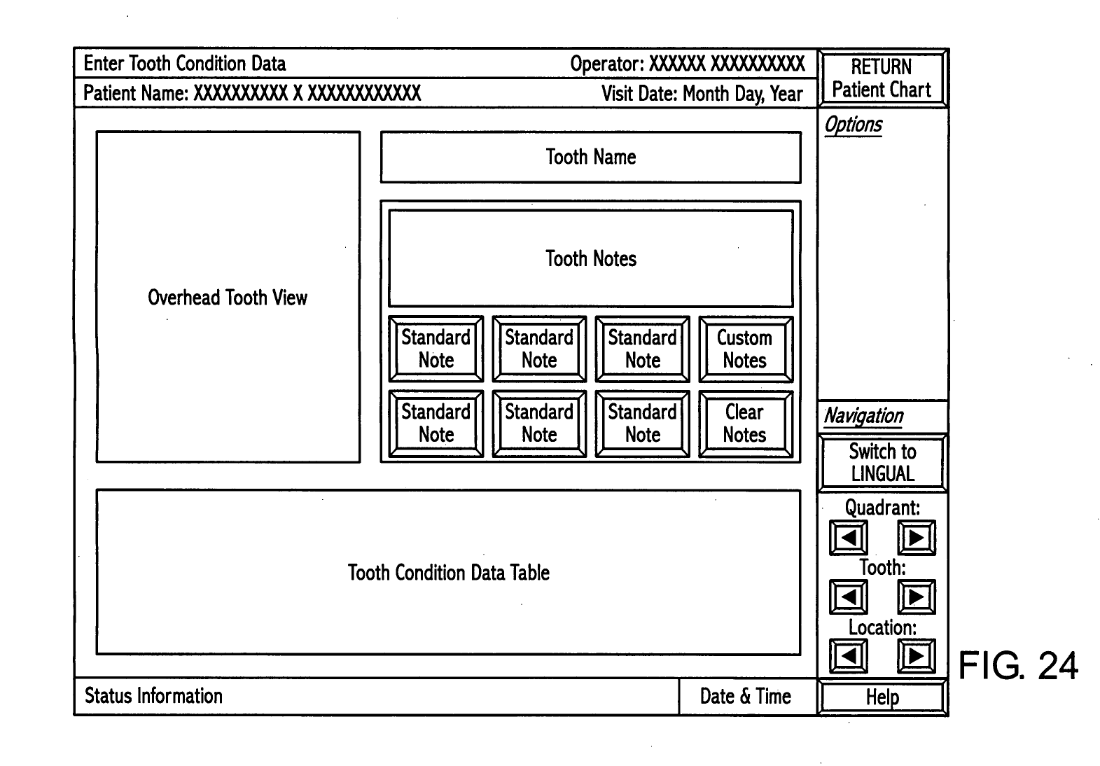

The Enter Tooth Condition Data Screen is used to enter tooth condition data for the patient according to the Tooth Condition Tables. All data displayed is retrieved from the internal database, and any data entered is stored in the internal database. FIG. 24 shows a notional view of the Enter Tooth Condition Data Screen.

The operator encounters the following fields and options in the Enter Tooth Condition Screen:
A. Overhead Tooth View: This is preferably a top-down image of teeth and is used to show to the operator the current tooth that is selected.
B. Teeth Name: Textual name of the currently selected tooth.
C. Tooth Notes: This field preferably shows the textual notes currently entered for this patient for this tooth.
   a. Standard Notes: In one aspect there are six buttons for entry of quick notes. These may be defined in the options screen.
   b. Custom Notes: The operator can enter in custom notes by clicking the Custom Notes button which takes them to a Keyboard Dialog.
   c. Clear Notes: This is preferably used to clear the notes field.
D. Tooth Condition Data Table: Tabulated tooth condition data for the currently selected quadrant according to tooth Condition Tables.
E. Navigation buttons:
   a. Switch to Facial/Lingual Button: These buttons preferably select between facial and lingual data to be presented on the Teeth Chart and the Tooth Condition Data Table.
   b. Tooth Selection Arrows: The operator can use these buttons to cycle which set of teeth is being highlighted in the Teeth Chart and therefore also tabulated in the Tooth Condition Data Table. The operator can cycle by location on tooth, by tooth, or by quadrant.
F. Return Button: This button preferably takes the operator back to the Chart Screen.
   Measure Pocket Depth Screen

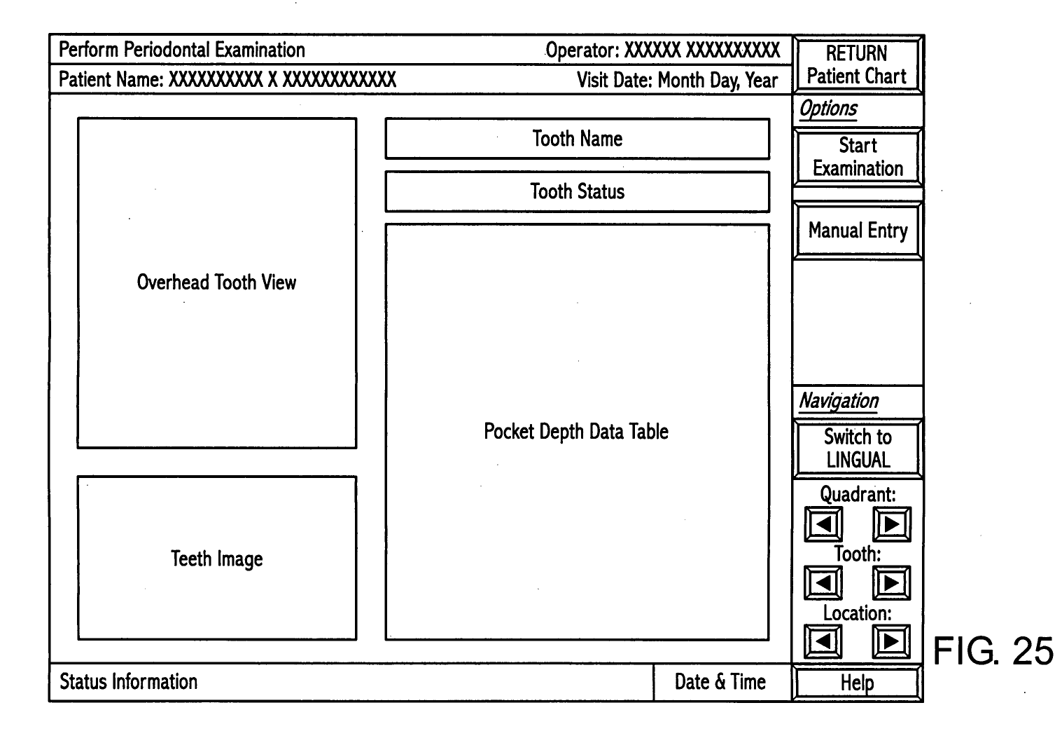

The Measure Pocket Depth Screen facilitates the collection of periodontal measurements. An operator can perform an automated scan examination, which uses the trigger device to trigger a pocket depth measurement and automatically proceed to the next tooth. An operator can also manually enter in data for a patient, either before, during, or after an automated examination. FIG. 25 shows a notional view of the Measure Pocket Depth Screen.

The operator encounters the following fields and options in the Measure Pocket Depth Screen:
A. Overhead Tooth View: This is preferably a top-down image of teeth and is used to show to the operator the current tooth that is selected.
B. Teeth Name: Textual name of the currently selected tooth.
C. Tooth Status: This static text preferably displays whether the currently selected tooth is missing.
D. Teeth Image: This is preferably a pictorial image of the mouth with the current tooth highlighted. This image is preferably used to help guide the operator during an examination.
E. Pocket Depth Data Table: This table preferably displays the pocket depth measurement results for each of the three locations (Mesial, Central, Distal), on a single side (Facial/Lingual), of the eight teeth of a quadrant.
F. Option Buttons:
   Start Examination: This button preferably toggles to start or stop an automated scan sequence. When starting the Calibration Screen is shown, this preferably guides the operator through calibration of the device. After calibration the operator preferably performs an automated scan.
   Manual Entry: This button preferably takes the operator to a keyboard entry dialog. The user can enter a depth manually for the current location. This is intended for entry of traditional probe measurements and preferably allows only a single unit (0-9) of data.
G. Navigation buttons:
   Switch to Facial/Lingual Button: These buttons preferably change the side of the tooth for the currently selected tooth.
   Tooth Selection Arrows: The operator can use these buttons to cycle the current selected tooth and location. The operator can cycle by location on tooth, by tooth, or by quadrant.
H. Return Button: This button preferably takes the operator back to the Chart Screen.
   Calibration Screen

The disposable shroud covering the ultrasonic transducer is intended to be replaced before each patient exam. Differences in individual shrouds due to manufacturing tolerances may cause slight differences in water flow and acoustic performance from exam to exam. Therefore, device calibration preferably occurs between data collection sessions.

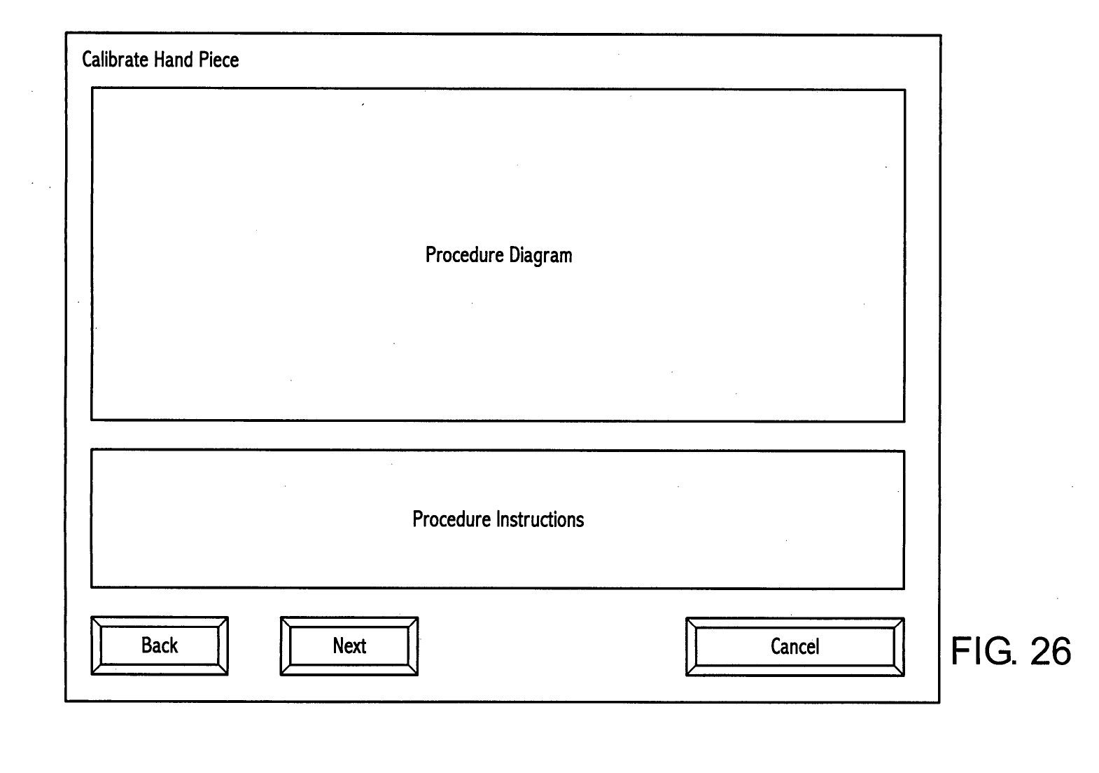

To assist the operator in calibrating the system, the application provides instructions to the operator via the Calibration Screen. Preferably, this screen is used to guide the operator through the attachment of a new hand piece 202 and the calibration of the periodontal system 200. The process may be broken down into distinct steps and the operator preferably guided through the steps by a calibration wizard. To navigate between steps in one aspect of the invention there is a Next and a Back button. FIG. 26 shows a notional view of the Calibration Screen.

In one embodiment of the invention, there are four steps in the calibration process. An instructional image for each step will appear in turn in the Procedure Display field. The steps in the calibration process are:
1. Attachment of New Hand piece 202: This step preferably explains that the operator is to remove a hand piece from the sterile packaging and how to attach it to the base piece.
2. Placement of Hand piece on Calibration Fixture: This step preferably shows the operator where to place the hand piece 202 on the device body to perform the calibration. In one aspect, there is an explanation that when the user presses the Next button that the device will take measurements to perform the calibration.
3. Perform Calibration: This step preferably shows the result of the calibration. Preferably, there is a statement whether the calibration was successful or not successful. If the calibration is not successful preferably there is a description of possible problems and remediation. In this case, the Next button is preferably disabled and the operator is typically required to return to the last step to redo the calibration.
4. Calibration Complete: This step preferably displays that the calibration is done and the operator can perform measurements on the patient.

The operator typically encounters the following fields and buttons in the Calibration Screen:
A. Procedure Diagram: This is the display for images to explain calibration steps.
B. Procedure Instruction: Textual information preferably with instructions for each step of the procedure.
C. Next & Back Button: These buttons preferably allow navigation through the calibration steps.
D. Cancel Button: This button preferably allows the operator to cancel out of the calibration if they have already performed a calibration on the current hand piece 202.
   View Tooth History Screen

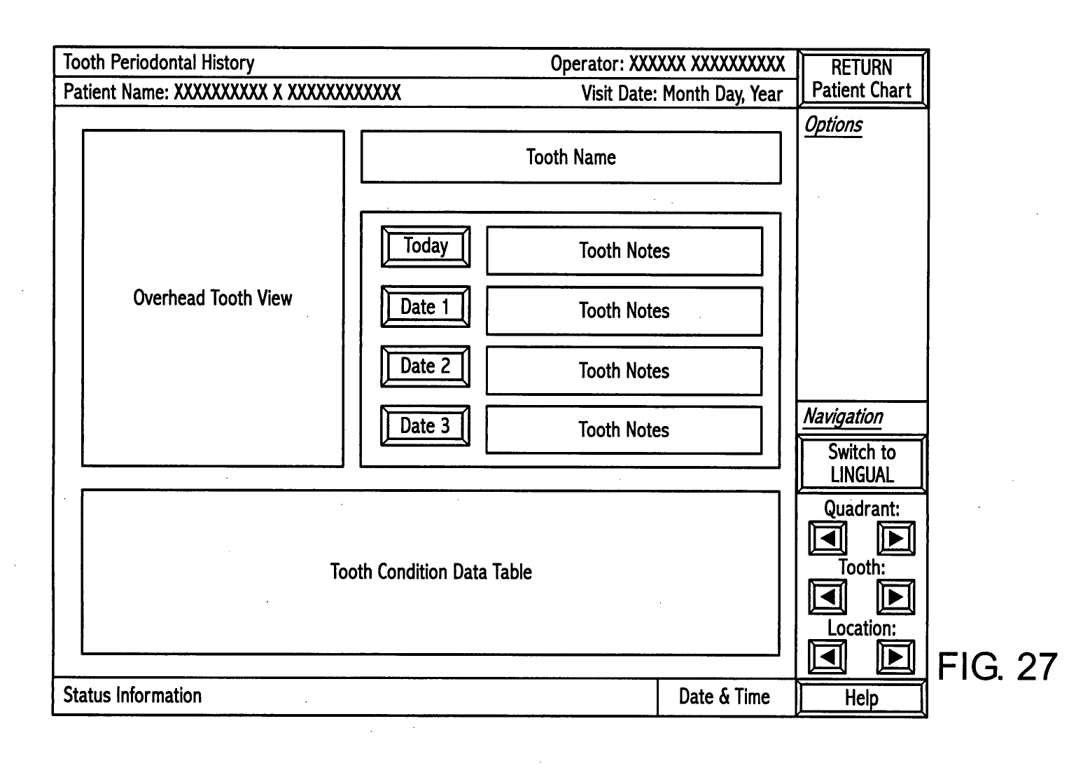

The View Tooth History Screen is a report screen that preferably presents to the operator data about a specific tooth, including current and past visit data. The operator on this screen can change between teeth. The past visits preferably are those that are selected on the Patient History Selection Screen. FIG. 27 shows a notional view of the View Tooth History Screen.

The operator encounters the following fields and options in the View Tooth History Screen:
A. Overhead Tooth View: Preferably, this is a top-down image of teeth and is used to show to the operator the current tooth that is selected.
B. Teeth Name: Textual name of the currently selected tooth.
C. Tooth Notes: This field preferably shows the textual notes entered for the currently selected tooth for the current and past visits.
D. Tooth Condition Data Table: Tabulated tooth condition data for the currently selected quadrant according to Tooth condition Tables.
E. Navigation buttons:
   a. Switch to Facial/Lingual Button: These buttons preferably change the side of tooth for the currently selected tooth.
   b. Tooth Selection Arrows: The operator can use these buttons to cycle the current selected tooth. The operator can cycle by location on tooth, by tooth, or by quadrant.
F. Return Button: This button preferably takes the operator back to the Chart Screen.
   View Full Patient Chart Screen

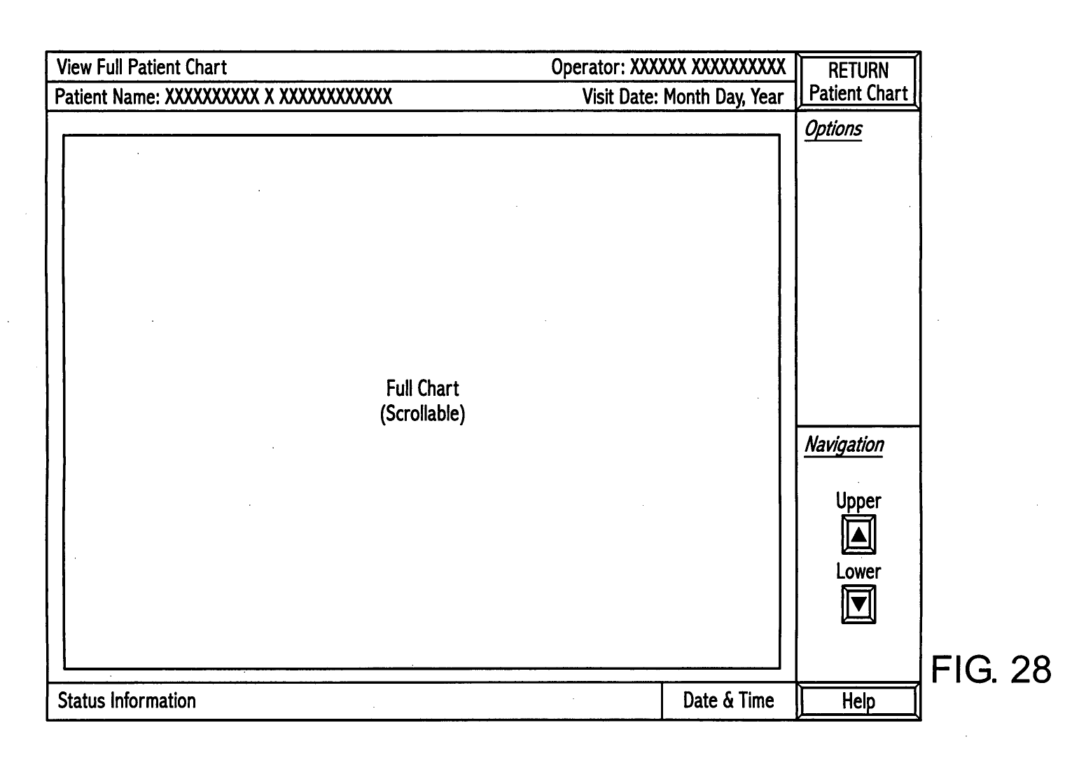

The View Full Patient Chart Screen is a report screen that preferably presents to the operator to view a full patient chart including periodontal and tooth condition data. FIG. 28 shows a notional view of the View Full Patient Chart Screen.

The operator encounters the following fields and options in the View Full Patient Chart Screen:
A. Full Chart: This image preferably displays a patient chart including periodontal depth measurements, tooth condition data, and graphical plots of depth overlays on teeth (similar to those of the Teeth Chart on the Chart Screen). In one aspect of the invention, only the upper or lower teeth are displayed at any one moment. In this aspect, the Navigation Buttons are used to switch between each teeth set. In another aspect of the invention, both the upper and lower teeth are displayed at the same time.
B. Navigation buttons:
   a. Upper/Lower Button: These buttons preferably switch the Full Chart between display of upper teeth and lower teeth.
C. Return Button: This button preferably takes the operator back to the Chart Screen.
   Patient History Selection Screen

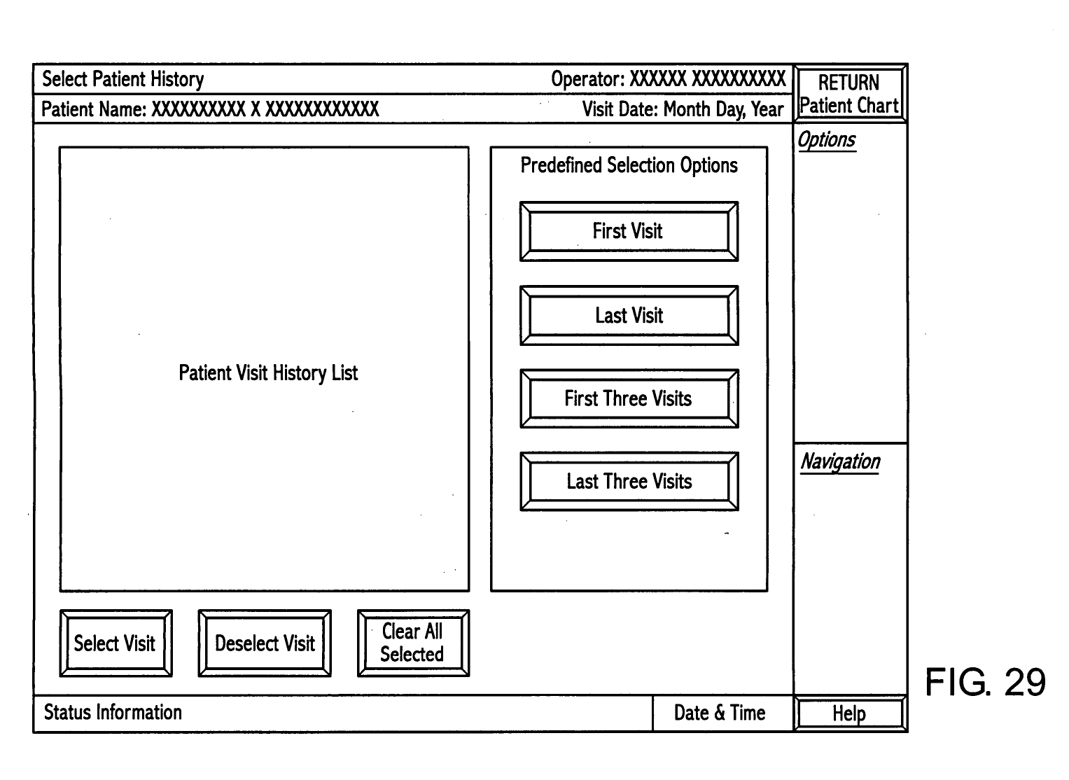

The Patient History Selection Screen allows the operator to select other patient visits. Preferably, these visits are those that are displayed as overlays on the Chart Screen, and in the View Tooth History Screen. FIG. 29 shows a notional view of the Patient History Selection Screen.

The operator encounters the following fields and options in the Patient History Selection Screen:
A. Patient Visit History List: This list box preferably shows a list of all visits for the currently selected patient. An operator clicks on individual visits in the field.
B. Data History Selection: This list box preferably contains options for quickly selecting patient visit histories. Options may include:
   Select, for example, the first three visit records. In other aspects of the invention, the number of visits selected may be more or less than three.
   Select, for example, the last three visit records. In other aspects of the invention, the number of visits selected may be more or less than three.
   Select the last visit.
   Select the first visit.
   Select a date range of prior tests.
C. Select Visit Button: Once a visit is selected in the Patient Visit History List, this button preferably adds the visit to the history selection.
D. Deselect Visit Button: This button preferably deselects the currently selected visit in the Patient Visit History List.
E. Clear All Selected Button: This button preferably deselects all selected visit in the Patient Visit History List.
F. Return Button: This button preferably takes the operator back to the Chart Screen.
   Help Screen

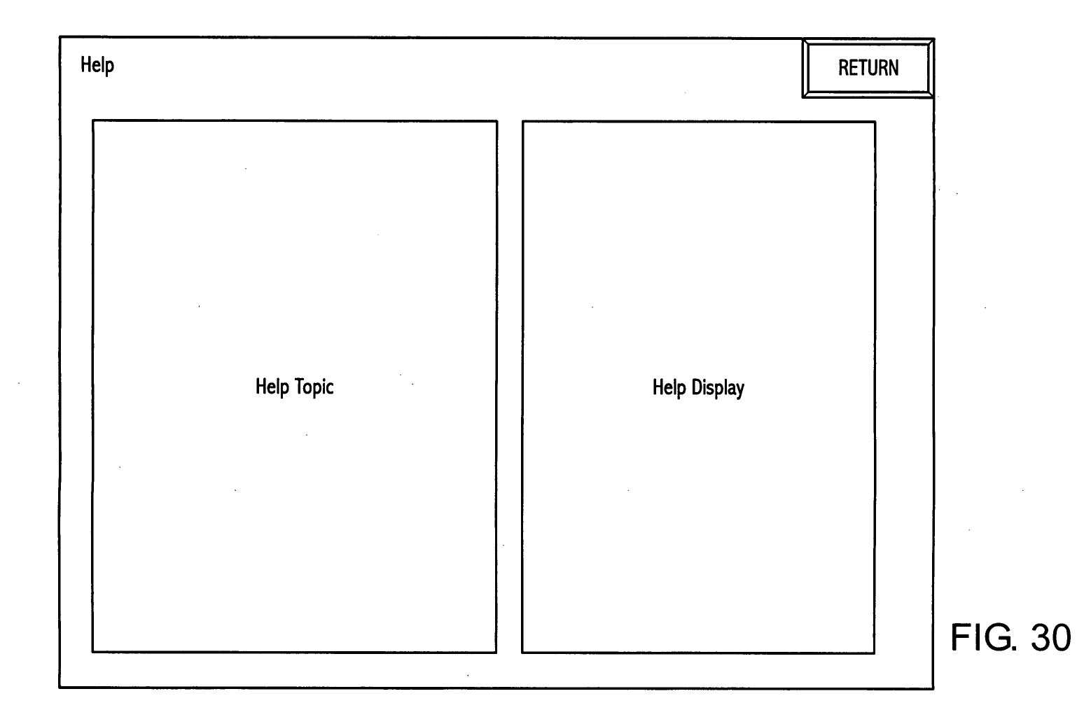

This screen has a list of topics that the operator can click on to get topic specific help. The information displayed preferably includes help on the device and educational material about the periodontal exam. FIG. 30 shows a notional view of the Help Screen.

The operator encounters the following fields and buttons in the Help Screen:
A. Help Topics: Preferably, this tree list displays the topics of interest that an operator can select.
B. Help Display: This field is preferably an HTML viewer that is used to display a combination of text and images that give specific help on a topic. The help is preferably stored internally as HTML code and graphics. The field need not be HTML, any suitable language may be used.
C. Return Button: This button preferably takes the operator back to the previous screen, where the operator initially called up the Help Screen.
   Additional Dialogs

Dialogs are typically used for specific user interface, both for data entry and to announce/warn the operator of a condition. Dialogs are generally considered modal, which means that once they are displayed the program preferably cannot continue until the user does some specific action to close the dialog such as clicking “OK” to a warning.

Keyboard Dialog

This dialog is preferably used throughout the program for text and numeric entry. It preferably contains all ten numbers (0-9), letters, a caps lock key, and a variety of special characters for entry. Preferably, there is a display for the data entered, an Enter button to accept the entry, and a Cancel button to cancel the entry.

Add/Edit Patient Dialogs

These dialogs preferably allow the operator to enter or edit information about a new or existing patient. The information that can be entered is:
Patient name
Dentist office
Dentist office patient ID
Date of birth
Gender
Address
Patient dental and medical history
Family history.
   Error Dialog

This dialog is preferably displayed if there is an error in the program. It contains a description of actions the operator can perform to remedy the error.

Find Patient Dialog

This dialog preferably contains a keyboard and is displayed to help the operator quickly find a patient in the list of patients by entering in the first few letters in the patient's name.

Data Acquisition

The data acquisition hardware includes the ultrasonic transducer 227, the pulser-receiver and the analog-to-digital converter (A/D card). Because water flow is preferable for recording of data, the relay that controls the water control solenoid valve preferably is also considered a data acquisition element.

When the operator of the periodontal system 200 enters calibration or clinical data acquisition commands through the user interface devices (the trigger device and the touchscreen 205), the user interface software preferably passes them on to the data acquisition software elements, which in turn issues appropriate commands to the hardware. The pulser-receiver, A/D card, and solenoid valve relay are preferably triggered or activated by logic level signals sent to them by the SBC through the parallel port. The pulser-receiver and the A/D card are preferably wired to the same parallel port channel, and are thus preferably triggered simultaneously. Sampled data is preferably passed from memory on the A/D card to the SBC memory, where it is accessible to the periodontal system 200 software application, over the A/D card's PCI header.

Data Acquisition and Analysis Sequence

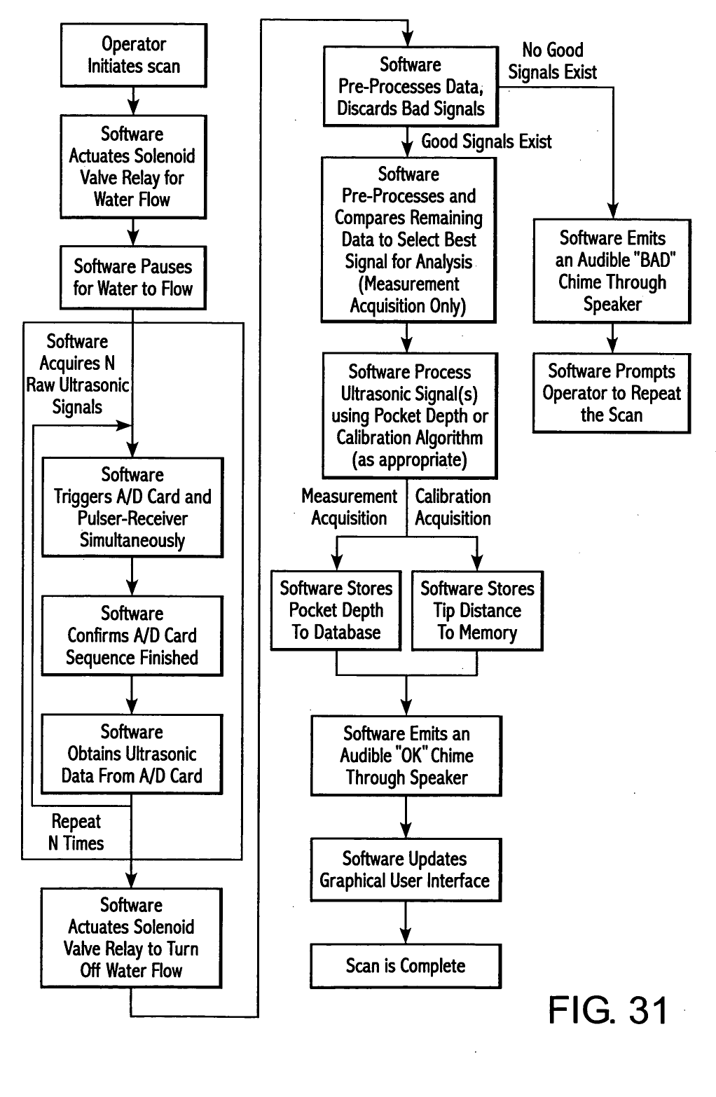

FIG. 31 is a diagram illustrating the sequence of the data acquisition and analysis events controlled by the software. This process is preferably executed each time the operator initiates a scan by pressing the trigger device.

Preferably, the data acquisition sequence of events is nearly identical for calibration and human in-use data acquisition. Once the operator initiates a scan either via the trigger mechanism, the solenoid valve relay preferably opens the valve to begin acoustic couplant water flow. Preferably, after a pause to allow flow stabilization, the application commands the data acquisition hardware to acquire N acoustic signals. Preferably each measurement acquisition transpires as follows:
the pulser/receiver is triggered and preferably “rings” the ultrasonic transducer with an electric pulse,
the ultrasonic transducer emits ultrasonic waves,
the ultrasonic transducer converts returning sonic echoes into an electric signal,
the pulser/receiver receives the signal from the transducer and conditions it for output,
the A/D card (preferably triggered with the pulser/receiver) records the returning signal to its memory, and
when N signals have been recorded, the software application preferably transfers all signals from A/D memory to system (SBC) memory for processing.

Preferably, once all N signals are in system memory, they are pre-processed twice. First they are all preferably compared to required criteria, and invalid signals are rejected. Second, the remaining signals are preferably compared to each other and the highest quality signal is selected for analysis. The analysis algorithm processes the signal to find the periodontal pocket depth which is then stored in the database.

The calibration acquisition sequence is nearly identical. Instead of N signals all being transferred at once, each signal is transferred and processed immediately after it is acquired (not shown in FIG. 31). The calibration signals are preferably all processed, and are not compared to each other. Preferably, the calibration signals are processed to determine the distance from transducer to the end of the hand piece tip. The average tip distance from the N signals is preferably used as the tip distance calibration parameter.

In both calibration and measurement acquisition cases, if data is collected and analyzed successfully a “successful” chime is preferably emitted from the periodontal system's 200 speaker. If an error occurs an “unsuccessful” chime preferably sounds and a message indicating the cause of the scan failure is shown on the display. Depending on the cause of the scan failure, the operator may then be prompted to repeat the scan.

The acquisition signal processing algorithm preferably converts the digital representation of the ultrasonic echo returning from the anatomical structure into a single measurement of the depth of the periodontal pocket, in units of millimeters with a resolution of 0.1 millimeters (0.0 mm-9.9 mm). Preferably, the calibration signal processing algorithm converts the digital representation of the ultrasonic echo returning from the calibration target into a single measurement of the distance from the transducer to the end of the hand piece tip in units of millimeters with a resolution of 0.1 millimeters.

Automatic Data Entry

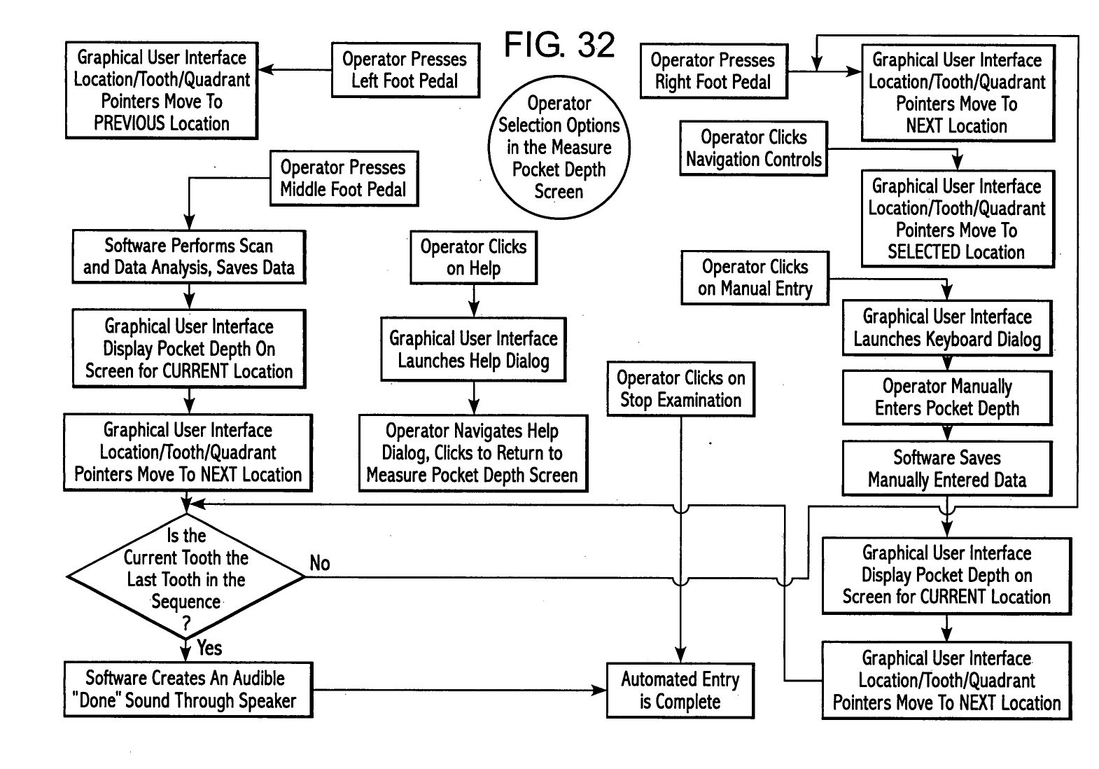

During a periodontal exam, the operator typically follows a pre-planned measurement route, collecting data as prompted by the device. The operator may skip measurement locations, or re-take a previously taken measurement using the controls on the Measure Pocket Depth Screen and the trigger device. FIG. 32 is a diagram of operator options during data collection, assuming a three-position foot-pedal is used as the triggering device. The operator can navigate to desired locations using the left and right foot pedals, and by using the location, tooth and quadrant navigation buttons on the Measure Pocket Depth Screen. In one aspect of the invention, pressing the middle foot pedal button initiates a scan. The operator can choose to measure the pocket depth manually and record the depth into the database using the Manual Entry button. The operator can click the Help button to enter the help area, and then return to the Measure Pocket Depth Screen. Finally, the operator can choose to end the examination by clicking the Stop Examination button.

Periodontal System 200 Database

All operator, patient and visit information used or collected by the periodontal system 200 is preferably contained in the periodontal system's 200 Database, which resides in the periodontal system 200. This database preferably contains both personal information for operators and patients (name, address, etc.) as well as clinical data (periodontal pocket depths, tooth condition notes, etc.). The periodontal system's 200 Database preferably includes six distinct tables, each discussed in detail below.

Because the periodontal system's 200 Database contains electronic personally identifiable health information (EPHI), privacy and security laws are preferably taken into account when this information is transmitted. A brief discussion of these issues is also contained below.

Operator Table

Table 4 summarizes the information stored in the periodontal system 200 Database for each operator. The OpID number is preferably assigned at the time of record creation, and is preferably sequential. Preferably, all fields are updated automatically as they are changed through the graphical user interface. Preferably, each time a new operator is given authorization to use a periodontal system 200 his or her information will become a new row record in the Operator Table. Although the data type of OpDOB is Date/Time, the time portion of OpDOB is typically never entered or accessed in the application, and can thus be ignored.

TABLE 4

| Field Name | Data Type | Description |
|---|---|---|
| OpID | AutoNumber | A unique record identifier, the |
| | | Operator's ID Number |
| OpLastName | Text | The Operator's Last Name |
| OpFirstName | Text | The Operator's First Name |
| OpMiddleName | Text | The Operator's Middle Name |
| OpAddress | Text | The Operator's Mailing Address |
| OpDOB | Date/Time | The Operator's DOB |
| OpPassword | Text | The Operator's Password |
| OpPreference1 | Number | The Operator's Data Collection |
| | | Route Preference |
| RecordCreated | Date/Time | The Date and Time the record |
| | | was created |
| RecordModified | Date/Time | The Date and Time the record |
| | | was modified |
| CreatorID | Number | The Operator ID of the record creator |
| ModifierID | Number | The Operator ID of the record modifier |

Patient Table

Table 5 summarizes the information preferably stored in the periodontal system 200 Database for each patient. The PatientID number is preferably assigned at the time of record creation, and is preferably sequential. Preferably, all fields are updated automatically as they are changed through the graphical user interface. The optional PatientFileID field may be used, for instance, to store the patient's ID number from the dental office's practice management software. Preferably, each time a patient is examined by a dental office using a periodontal system 200 for the first time, his or her information will become a new row record in the Patient Table. Typically the data type of PatientDOB is Date/Time, the time portion of PatientDOB is never entered or accessed in the application, and can thus be ignored.

TABLE 5

| Field Name | Data Type | Description |
|---|---|---|
| PatientID | AutoNumber | A unique record identifier, the Patient's |
| | | ID Number |
| PatientLastName | Text | The Patient's Last Name |
| PatientFirstName | Text | The Patient's First Name |
| PatientInitial | Text | The Patient's Middle Name |
| PatientGender | Number | Patient Gender: 0 = Male, 1 = Female |
| PatientAddress | Text | The Patient's Mailing Address |
| PatientDOB | Date/Time | The Patient's DOB |
| PatientFileID | Text | An optional additional patient identifier |
| | | for use in the office |
| RecordCreated | Date/Time | The Date and Time the record |
| | | was created |
| RecordModified | Date/Time | The Date and Time the record |
| | | was modified |
| CreatorID | Number | The Operator ID of the record creator |
| ModifierID | Number | The Operator ID of the record modifier |

Visit Table

Table 6 summarizes the information stored in the periodontal system 200 Database for each patient visit. The VisitID number is preferably assigned at the time of record creation, and is preferably sequential. Preferably, all fields are updated automatically as they are changed through the graphical user interface. The VisitDataFileName field preferably contains the directory pathway to a file containing the raw periodontal data for the visit. Preferably, the data is not itself stored in the database to prevent the database from growing too large. Preferably, each time a periodontal exam is begun it will result in the creation of a new visit row record in the Visit Table.

TABLE 6

| Field Name | Data Type | Description |
|---|---|---|
| VisitID | AutoNumber | A unique record identifier, the |
| | | Visit ID Number |
| PatientID | Number | The Patient's ID Number |
| OpID | Number | The Operator's ID Number |
| VisitDate | Date/Time | The Date of the Visit |
| VisitDataFileName | Text | The directory path to the raw |
| | | periodontal data file |
| RecordCreated | Date/Time | The Date and Time the record |
| | | was created |
| RecordModified | Date/Time | The Date and Time the record |
| | | was created |
| CreatorID | Number | The Operator ID of the record creator |
| ModifierID | Number | The Operator ID of the |
| | | record modifier |
| VisitNotes | Text | The Notes entered by the examiner |
| VisitStatus | Number | The overall exam status, default of |
| | | 0 means untaken |

Visit Tooth Condition Table

Table 7 summarizes the Tooth Condition information stored in the periodontal system 200 Database for each visit. The DataID number is preferably assigned at the time of record creation, and is preferably sequential. The VisitID field preferably contains the Visit ID number of the periodontal exam during which the Tooth Condition was recorded. Preferably, all fields are updated automatically as they are changed through the graphical user interface. In one aspect of the invention, the fields TIC through T32C each contain five comma-separated digits, each with a value of 0 or 1. Each digit preferably refers to the presence (value of 1) or absence (value of 0) of a certain tooth condition. In this aspect of the invention, the five digits refer in order to: missing tooth, suppuration, bleeding, mobility, and edema. In another aspect of the invention, additional digits may be assigned to refer to a tooth with a cap or a re-implanted tooth. Preferably, each time a periodontal exam is begun it will result in the creation of a new tooth condition row record in the Tooth Condition Table.

TABLE 7

| Field Name | Data Type | Description |
|---|---|---|
| DataID | AutoNumber | A unique record identifier, the |
| | | data ID Number |
| Visit ID | Number | Related VisitID record |
| RecordCreated | Date/Time | The Date and Time the record |
| | | was created |
| RecordModified | Date/Time | The Date and Time the record |
| | | was created |
| CreatorID | Number | The Operator ID of the record creator |
| ModifierID | Number | The Operator ID of the record modifier |
| T1C | Text | A series of five comma-separated digits |
| | | with values of one or zero |
| . . . | . . . | . . . |
| T32C | Text | A series of five comma-separated digits |
| | | with values of one or zero, representing |
| | | tooth condition |

Visit Depth Table

Table 8 summarizes the Depth measurement information stored in the periodontal system's 200 Database for each visit. The DataID number is preferably assigned at the time of record creation, and is preferably sequential. The VisitID field preferably contains the Visit ID number of the periodontal exam during which the Depth measurements were recorded. Preferably all fields are updated automatically as they are changed through the graphical user interface. In one aspect of the invention, the fields T1D through T32D each contain 24 comma-separated numbers. These 24 numbers preferably represent four values, measured or assessed by the operator at the time of the exam, for each of six locations around a tooth. The four values preferably refer in order to: periodontal pocket depth, level of furcation, level of recession, and status. Preferably each time a periodontal exam is begun it will result in the creation of a new depth row record in the Depth Table. In another embodiment of the invention, measurement are taken in the interdental space between teeth rather than in six locations around a tooth. In this embodiment, the numbers represent values taken behind the papilla and are associated with the deterioration periodontal tissue (gum 102, periodontal ligament 103, 126, and the specula of bone between the teeth) due to periodontal disease. Further, additional numbers may be assigned to record measurements of teeth that have been capped or re-implanted.

TABLE 8

| Field Name | Data Type | Description |
|---|---|---|
| DataID | AutoNumber | A unique record identifier, the |
| | | data ID Number |
| Visit ID | Number | Related VisitID record |
| RecordCreated | Date/Time | The Date and Time the record |
| | | was created |
| RecordModified | Date/Time | The Date and Time the record |
| | | was created |
| CreatorID | Number | The Operator ID of the record creator |
| ModifierID | Number | The Operator ID of the record modifier |
| T1D | Text | A series of 24 comma-separated |
| | | numbers representing measured values |
| | | at that tooth |
| . . . | . . . | . . . |
| T32D | Text | A series of 24 comma-separated |
| | | numbers representing measured values |
| | | at that tooth |

Tooth Notes Table

Table 9 summarizes the information stored in the periodontal system's 200 Database for each tooth note recorded during a periodontal visit. The NoteID number is preferably assigned at the time of record creation, and is preferably sequential. The VisitID field preferably contains the Visit ID number of the periodontal exam during which the note was recorded. The TNum field preferably contains the tooth number for which the note was created. Preferably, all fields are updated automatically as they are changed through the graphical user interface. Preferably the Note field is simply a text string containing the note recorded by the operator. Preferably each time a note is recorded it will result in the creation of a new note row record in the Tooth Note Table.

TABLE 9

| Field Name | Data Type | Description |
|---|---|---|
| NoteID | AutoNumber | A unique record identifier, the |
| | | data ID Number |
| VisitID | Number | Related VisitID record |
| RecordCreated | Date/Time | The Date and Time the record |
| | | was created |
| RecordModified | Date/Time | The Date and Time the record |
| | | was modified |
| CreatorID | Number | The Operator ID of the record creator |
| ModifierID | Number | The Operator ID of the record modifier |
| Tnum | Number | The Tooth to which the note attaches |
| Note | Text | The Text of the Notes |

HIPAA Conformance

Preferably because the periodontal system 200 collects, stores and transmits personal health information, it should conform to HIPAA (Health Insurance Portability and Accountability Act) regulations. These regulations require that measures be taken to ensure the privacy and security of personal health information. That information must also be available for authorized transmission in an industry-standard format to facilitate administrative simplification.

To comply with HIPAA, any personally-identifiable health information that is transmitted by the periodontal system 200 is preferably encrypted. Typically, encryption should be limited where possible by sending clinical data only, and omitting personal identifiers such as dates of birth, addresses, and names of patients and operators. Data will may also be available for transmission in a format compliant with HIPAA standards. Flexibility exists in the strategy employed to meet these evolving standards.

External Interface

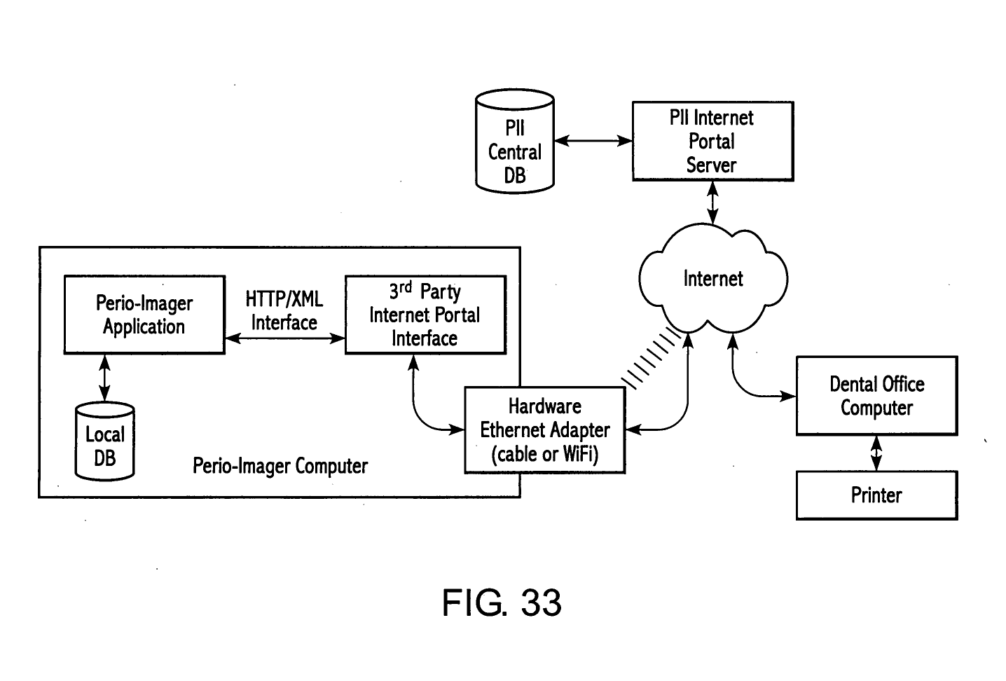

The periodontal system 200 can communicate with the external world over the Internet. This is preferable in order to synchronize the database in each periodontal system 200 in an office, to allow online software upgrades, to print reports of periodontal data, and perform other activities. FIG. 33 below depicts the primary software and hardware-components involved in the periodontal system 200 external communications.

The preferred communications strategy is to have each periodontal system 200 communicate individually with and only with the Portal. The Internet Portal preferably has access to a central database which, for each client office, stores all previously measured data and all recorded patient visit and operator information that originated from that office. At start-up and/or prior to an exam, the periodontal system 200 preferably requests an update to its local database from the Internet Portal. This preferably synchronizes its database with the office central database on the Internet Portal. Preferably, this allows a patient to be examined using any periodontal system 200 device in a dental office. It also preferably gives operators access to all patient periodontal information, regardless of which device(s) have been used to examine the patient in the past. Preferably any dental office healthcare provider will be able to view or print periodontal reports, originating from their office, from a standard web browser by logging into the Internet Portal.

The periodontal system 200 preferably comes standard with a Category-5 cable Ethernet adapter installed. An optional wireless Ethernet adapter is also available.

The periodontal system's 200 software application preferably communicates with the Internet Portal via a third-party interface application. This interface application and the Internet Portal and are described below.

Internet Portal

Communication with the Internet Portal preferably accommodate the transferal and synchronization of database information (operator, patient and visit information) between the local (device) and central databases. This also is preferably the pathway for the transferal of usage and account information and software updates. Patient and operator information can be entered directly into the periodontal system's 200 devices, or, through a web browser, directly into the dental office's central database for subsequent download by the individual devices. Entering information for a new patient into the periodontal system's 200 Database via web browser and the Internet Portal is convenient, for instance, while entering the same information into the dental office practice management software application. All information transmission to and from the Internet Portal is preferably done in compliance with HIPAA privacy and security rules.

Internet Portal Interface Application

The Internet Portal Interface application facilitates two way communications between the periodontal system's 200 software application and the Internet Portal. It is preferably HTTP/XML based. Coordination between dental office practice management software packages and the periodontal system's 200 software application, if any, occurs via the Portal Interface Application.

Illustrative Example

Peak Detection

Peaks were detected by first selecting a signal threshold. The threshold selected was the value of the 90^{th} percentile of the waveform values. In other words, the level where only 10% of the signal is greater than the threshold. Then, local maxima were found by comparing each value in the waveform with values up to one nominal wavelength on either side, i.e. five points before and after. If the value was greater than the values to either side, it was designated as a maxima. Finally, the significance of the peak was determined by integrating over the same range. If the average value of the range was greater than the threshold value, then the maxima was determined to be a significant peak.

Peak Discrimination

The largest significant peak after the end of the tip was determined to correspond to the reflection off of the gum. The last significant and consistent peak (i.e. the peak is present in 60% of the replicate scans) within the measurement range (0 to 10 mm) was determined to be the bottom of the pocket. (see FIG. 39). Additional data, not shown, establish the distance from the gum line to the cemento-enamel junction. Thus, it is possible to determine the attachment loss by subtracting the distance from the gum line to the cemento-enamel junction from the distance of the gum line to the bottom of the pocket.

The foregoing description of the invention has been presented for purposes of illustration and description. It is not intended to be exhaustive or to limit the invention to the precise form disclosed, and modifications and variations are possible in light of the above teachings or may be acquired from practice of the invention. The drawings and description were chosen in order to explain the principles of the embodiments of the invention and its practical application. It is intended that the claims and terms thereof be interpreted to protect the invention to the broadest extent permitted by the prior art and the applicable law. Moreover, features described in connection with one embodiment of the invention may be used in conjunction with other embodiments, even if not explicitly stated above.

What is claimed is:

1. A system for detecting and measuring periodontal tissue destruction related to periodontal disease comprising:
a hand piece having an ultrasonic transducer, and an acoustic lens;
a controller unit having discrimination analysis software, the discrimination analysis software comprising a wavelet algorithm; and
a fluid supply.

2. The system of claim 1, wherein the fluid supply comprises a fluid reservoir.

3. The system of claim 1, wherein the system is adapted to measures periodontal pocket depth.

4. The system of claim 1, further comprising an input device.

5. The system of claim 4, wherein the input device comprises a touch screen, a keyboard, or a mouse.

6. The system of claim 1, further comprising a display, the display capable of displaying an image of a tooth and illustrating periodontal measurements in combination with the image of the tooth.

7. The system of claim 1, further comprising software, wherein the software converts an analog signal from the hand piece to digital data.

8. The system of claim 7, further comprising software to create dental images.

9. The system of claim 1, wherein the software includes discrimination analysis algorithm adapted to identify peaks associated with periodontal features.

10. The system of claim 1, wherein the system is adapted to measure the differential depth between the cemento-enamel junction of a tooth and the bottom of a periodontal pocket.

11. The system of claim 1, wherein the system is adapted to measure the differential depth between the gum line of a tooth and the bottom of a periodontal pocket.

12. The system of claim 10, wherein the system is adapted to measure the differential depth a periodontal pocket with an accuracy between about 0.1 mm and 0.5 mm.

13. The system of claim 12, wherein the hand piece adapted to measure the depth with an accuracy of about 0.2 mm.

14. The system of claim 13, wherein the hand piece adapted to measure the depth with an accuracy of about 0.1 mm.

15. The system of claim 1, wherein the system adapted to detect calculus.

16. The system of claim 1, wherein the fluid supply comprises an anti-bacterial agent, a germicidal agent or both.

17. The system of claim 1, further comprising a trigger to activate the ultrasonic transducer, the fluid supply, or both.

18. The system of claim 17, wherein the trigger comprises at least one foot pedal, or voice activation software.

19. The system of claim 1, wherein the system is connected to the Internet.

20. The system of claim 19, wherein the connection is accomplished by wire or wirelessly.

21. The system of claim 1, wherein periodontal measurements can be made with a single operator.

22. The system of claim 1, wherein the system stores periodontal measurements in an electronic medical record.

23. The system of claim 22, wherein the electronic medical record is HIPAA compliant.

24. The system of claim 19, further comprises enterprise portal software.

25. The system of claim 24, wherein the system adapted to be upgraded via Internet.

26. The system of claim 25, further comprising a tone generator, wherein the tone generator generates one tone to indicate a successful test and a second tone to indicate an unsuccessful test.

27. A dental system for detecting and measuring comprising:
a dental tool having an ultrasonic transducer; and a controller unit having discrimination analysis software, the discrimination analysis software comprising a wavelet algorithm,
the dental tool adapted for signal communication with the controller unit,
wherein the ultrasonic transducer is adapted to produce an ultrasonic signal capable of being transmitted thorough a fluid signal coupler to produce dental signals for processing by the discrimination analysis software.

28. The system of claim 27, further comprising a fluid supply.

29. The system of claim 27, wherein the system is adapted to measure periodontal pocket depth.

30. The system of claim 27, where the dental signal is a reflection from a periodontal feature.

31. The system of claim 27, further comprising a display, the display is capable of displaying an image of a tooth and illustrating periodontal measurements in combination with the image of the tooth.

32. The system of claim 27, further comprising software, wherein the software converts an analog signal from the hand piece to digital data.

33. The system of claim 32, further comprising software to create dental images.

34. The system of claim 27, wherein the software includes discrimination analysis algorithm adapted to identify peaks associated with periodontal features.

35. The system of claim 27, wherein the system is adapted to measure the differential depth between the cemento-enamel junction of a tooth and the bottom of a periodontal pocket.

36. The system of claim 27, wherein the system is adapted to measure the differential depth between the gum line of a tooth and the bottom of a periodontal pocket.

37. The system of claim 35, wherein the system is adapted to measure the differential depth a periodontal pocket with an accuracy between about 0.1 mm and 0.5 mm.

38. The system of claim 37, wherein the hand piece is adapted to measure the depth with an accuracy of about 0.2 mm.

39. The system of claim 38, wherein the hand piece is adapted to measure the depth with an accuracy of about 0.1 mm.

40. The system of claim 27, wherein the system is adapted to detect calculus.

41. A method of detecting and measuring periodontal tissue destruction related to periodontal disease comprising:
filing a periodontal pocket with a fluid capable of propagating sound waves;
transmitting at least one sound wave into the periodontal pocket;
sensing at least one return sound wave from the periodontal pocket; and
determining the depth of the pocket by measuring the time it takes the at least one transmitted sound wave to traverse the periodontal pocket and return.

42. The method of claim 41, further comprising storing the pocket depth as periodontal data.

43. The method of claim 42, wherein further comprising comparing pocket depths against stored periodontal data.

44. The method of claim 41, wherein the periodontal data is stored in an electronic medical record.

45. The method of claim 41, further comprising generating 3-D images of a patient's teeth.

46. The method of claim 41, further comprising sending data to a remote display.

47. The method of claim 41, further comprising storing the periodontal data at a remote site.

48. The method of claim 47, wherein the stored periodontal data is accessed via the Internet.

49. The method of claim 41, including the step of determining the location of the cemento-enamel junction.

50. The method of claim 41, further comprising including an antibacterial agent, a germicidal agent, or both in the fluid.

51. The method of claim 41, further comprising transmitting at least one sound wave in the interdental space between teeth.

52. The method of claim 51, further comprising determining the deterioration of the specula of bone between teeth.

53. The method of claim 41, comprising digitally overlaying periodontal measurements onto a digitally generated image of a tooth.

54. The method of claim 41, further comprising using a discrimination analysis software to identify periodontal features.

55. The method of claim 54 wherein the discrimination analysis software comprising a wavelet algorithm

56. The method of claim 55, further comprising identifying calculus on a tooth.

57. The method of claim 56, further comprising retesting a patient after a calculus treatment to evaluate the efficacy of the treatment.

58. The method claim 41, further comprising adding medication to the fluid.

59. The method of claim 47, further comprising monitoring progress of a course of treatment.

60. The method of claim 41, further comprising calibrating the system.

61. The method of claim 60, wherein the step of calibrating comprises testing software, calibrating a hand piece, testing a transducer, testing acoustic lens, or any combination thereof.

62. The method of claim 60, wherein calibration comprises measuring the depth of a known cavity.

63. The method of claim 44, further comprising forwarding the electronic medical record to at least one periodontal expert for consultation.

64. The method of claim 63, wherein the periodontal expert recommendations are sent to a third party or to the treating medical profession.

65. The method of claim 47, further comprising monitoring a dentist or hygienist.

66. The method of claim 47, further comprising identifying a patient being treated by dentist that should be treated by periodontist.

67. The method of claim 56, wherein the discrimination analysis software is upgraded via download from the Internet.

68. The method of claim 47, further comprising showing the electronic medical record to a patient.

69. The method of claim 41, wherein the flow of fluid is laminar.

70. The method of claim 70, wherein a Reynolds number is less than approximately 1000.

71. The method of claim 70, wherein a volumetric flow rate is between approximately 10 to 15 mL/min.

72. The method of claim 41, wherein minor changes in pocket depth are identified.

73. The method of claim 72, wherein the minor change comprises a change of pocket depth of approximately 0.2 to 0.5 mm.

74. The method of claim 73, wherein the minor change comprises a change of pocket depth of approximately 0.1 to 0.2 mm

75. The method of claim 41, wherein the method is performed with a hand piece having a fluid flow path designed to facilitate flushing of air bubbles.

76. The method of claim 41, further comprising transmitting at least one sound wave on the facial side of a tooth and at least one sound wave on the lingual side of the tooth.

77. The method of claim 76, further comprising transmitting at least three sound waves on both the facial and lingual sides of the tooth.

78. The method of claim 77, further comprising transmitting at least 12 sound waves on both the facial and lingual sides of the tooth.

79. A method of performing periodontal examinations comprising:
providing dentists or dental hygienists with at least one ultrasonic periodontal system; and
charging the dentist or dental hygienist per visit of a patient.

80. The method of claim 79, wherein the ultrasonic periodontal system is connected to the Internet with enterprise portal software.

81. The method of claim 80, wherein the ultrasonic periodontal system is connected to a third party for data backup.

82. The method of claim 81, further comprising forwarding dental records to at least one periodontal expert for consultation.

83. The method of claim 82, wherein periodontal expert recommendations are sent to third party or to a treating medical profession.

84. The method of claim 80, further comprising monitoring a dentist or dental hygienist.

85. The method of claim 80, further comprising identifying a patient being treated by dentist that should be treated by periodontist.

86. The method of claim 80, wherein the ultrasonic periodontal system comprises software that can be upgraded via a download from the Internet.

87. A dental method of detecting and measuring comprising:
providing a dental tool capable of propagating sound waves through a signal coupler;
transmitting at least one sound wave through the signal coupler onto a dental feature;
sensing at least one return sound wave from the dental feature; and
determining a characteristic of the dental feature by measuring the time it takes the at least one transmitted sound wave to return.

88. The method of claim 87, wherein the dental feature is a periodontal pocket and the characteristic is a pocket depth.

89. The method of claim 88, further comprising storing the pocket depth as periodontal data and comparing pocket depths against stored periodontal data.

90. The method of claim 87, wherein the periodontal data is stored in an electronic medical record.

91. The method of claim 87, further comprising generating 3-D images of a patient's teeth.

92. The method of claim 87, further comprising sending data to a remote display.

93. The method of claim 87, further comprising storing the periodontal data at a remote site.

94. The method of claim 87, wherein the stored periodontal data is accessed via the Internet.

95. The method of claim 87, including the step of determining the location of the cemento-enamel junction.

96. The method of claim 87, further comprising including an antibacterial agent, a germicidal agent, or both in the fluid.

97. The method of claim 87, further comprising transmitting at least one sound wave in the interdental space between teeth.

98. The method of claim 97, further comprising determining the deterioration of the specula of bone between teeth.

99. The method of claim 87, comprising digitally overlaying periodontal measurements onto a digitally generated image of a tooth.

100. The method of claim 87, further comprising using a discrimination analysis software to identify periodontal features.

101. The method of claim 100, wherein the discrimination analysis software comprising a wavelet algorithm

102. The method of claim 101, further comprising identifying calculus on a tooth.

103. The method of claim 102, further comprising retesting a patient after a calculus treatment to evaluate the efficacy of the treatment.
